SEQUENCE LISTING (11 sequences)

SEQ ID NO: 1 - DNA, 1754 bp, Rattus norvegicus
cccccgggggaggtgcgagagggctggaaaggacaggtccgggcagcgatcgggggttggcatcagttcgctcacccttcgagaggcagatcgctcttgtccgcaatcttcgctgaccgcgctagctgcggcttctgtgctccttcgccgaacctcatcaaccagcgtcctggcgtctgacctcgccatgcctagcctttgggatcgtttctcgtcctcctcttcctcttcgtcctcgtcccgaactccggccgctgatcggccgccgcgctccgcctgggggtctgcggccagagaagagggccttgaccgctgcgcgagcctggagagctcggactgcgagtccctggacagcagcaacagtggctttgggccggaggaagactcctcatacctggatggggtgtctctgcctgactttgagctgctcagtgaccccgaggatgagcacctgtgtgccaacctgatgcagctgctgcaggagagcctgtcccaggcgcgattgggctcgcggcgccctgcgcgcctgctgatgccgagccagctgttgagccaggtgggcaaggaactcctgcgcctggcgtacagcgagccgtgcggcctgcggggggcactgctggacgtctgtgtggagcaaggcaagagctgccatagtgtggctcagctggctctggaccccagtctagtgcccacctttcagttgaccctggtgctgcgtctggactctcgcctctggcccaagatccagggcctgttgagttctgccaactcttccttggtccctggttacagccagtccctgacgctgagcaccggcttcagagtcatcaaaaagaaactctacagctccgagcagctgctcattgaagagtgttgaacttcgtcctggaggggggccgcactgccccccaaagtggagacaaggaatttctgtggtggagacccgcaggcaaggactgaaggactgtcccctgtgttagaaaactgacaatagccaccggaggggcgcagggccaggtgggagaaggaagtgttgtccaggaagtctctaggttgtgtgcaggtggccccctgttggggcacatgcccctcagtactgtagcatgaaacaaaggcttcggagccacacaggcttctggctggatgtgtatgtagcatgtatcttattaatttttgtattactgacaagttacaacagcagttgtgggccagagtcagaagggcagctggtctgcactggcctctgcccgggctgtgtgctggggggaggcggggggaggtctccgacagtttgtcgacagatctcatggtctgaaaggaccgagcttgttcgtcgtttggtttgtatcttgttttgggggtggggtggggggatcggagcttcactactgacctgttcgaggcagctatcttacagactgcatgaatgtaagaataggaagggggtgggtgttaggatcatttgggatcttcaacacttgaaacaaaataacaccagggagctgcatcccagcccatcccggtgccggtgtactggaggagtgaactgtgaggggatggggctgaggggggtggggggctggaaccctctcccccagaggagcgccacctgggtcttccatctagaactgtttacatgaagatactcacggttcatgaatacacttgatgttcaagtactaagacctatgcaatatttttacttttctaataaacatgtttgttaaaacaaaaaaaaaaaaaaaaaaaaaaaa

SEQ ID NO: 2 - DNA, 1782 bp, Homo sapiens
tttggccctcgaggccaagaattcggcacgagggggggaggtgcgagcgtggacctgggacgggtctgggcggctctcggtggttggcacgggttcgcacacccattcaagcggcaggacgcacttgtcttagcagttctcgctgaccgcgctagctgcggcttctacgctccggcactctgagttcatcagcaaacgccctggcgtctgtcctcaccatgcctagcctttgggaccgcttctcgtcgtcgtccacctcctcttcgccctcgtccttgccccgaactcccaccccagatcggccgccgcgctcagcctgggggtcggcgacccgggaggaggggtttgaccgctccacgagcctggagagctcggactgcgagtccctggacagcagcaacagtggcttcgggccggaggaagacacggcttacctggatggggtgtcgttgcccgacttcgagctgctcagtgaccctgaggatgaacacttgtgtgccaacctgatgcagctgctgcaggagagcctggcccaggcgcggctgggctctcgacgccctgcgcgcctgctgatgcctagccagttggtaagccaggtgggcaaagaactactgcgcctggcctacagcgagccgtgcggcctgcggggggcgctgctggacgtctgcgtggagcagggcaagagctgccacagcgtgggccagctggcactcgaccccagcctggtgcccaccttccagctgaccctcgtgctgcgcctggactcacgactctggcccaagatccaggggctgtttagctccgccaactctcccttcctccctggcttcagccagtccctgacgctgagcactggcttccgagtcatcaagaagaagctgtacagctcggaacagctgctcattgaggagtgttgaacttcaacctgagggggccgacagtgccctccaagacagagacgactgaacttttggggtggagactagaggcaggagctgagggactgattcctgtggttggaaaactgaggcagccacctaaggtggaggtgggggaatagtgtttcccaggaagctcattgagttgtgtgcgggtggctgtgcattggggacacatacccctcagtactgtagcatgaaacaaaggcttaggggccaacaaggcttccagctggatgtgtgtgtagcatgtaccttattatttttgttactgacagttaacagtggtgtgacatccagagagcagctgggctgctcccgccccagcccggcccagggtgaaggaagaggcacgtgctcctcagagcagccggagggaggggggaggtcggaggtcgtggaggtggtttgtgtatcttactggtctgaagggaccaagtgtgtttgttgtttgttttgtatcttgtttttctgatcggagcatcactactgacctgttgtaggcagctatcttacagacgcatgaatgtaagagtaggaaggggtgggtgtcagggatcacttgggatctttgacacttgaaaaattacacctggcagctgcgtttaagccttcccccatcgtgtactgcagagttgagctggcaggggaggggctgagagggtgggggctggaacccctccccgggaggagtgccatctgggtcttccatctagaactgtttacatgaagataagatactcactgttcatgaatacacttgatgttcaagtattaagacctatgcaatattttttacttttctaataaacatgtttgttaaaacaaaaaaaaaaaaaaaaaa

SEQ ID NO: 3 - DNA, 1900 bp, Rattus norvegicus
ccatccctcataggactaattatagggttgggggggccgcccccccaggttcgagtggcgatgggccgcggctggggcttgctcgtcggactcttgggcgtcgtgtggctgctgcggtcgggccagggcgaggagcagcagcaggagacagcggcacagcggtgtttctgtcaggttagtggttacctggatgactgtacctgtgatgtcgagaccatcgataagtttaataactacagacttttcccaagactacaaaagctccttgaaagtgactactttagatactacaaggtaaacttgaggaagccatgtcctttctggaatgacatcaaccaatgtggaagaagagactgtgctgtcaaaccctgccattctgatgaagtccctgatggaattaagtctgcgagctacaagtattccaaggaagccaacctccttgaggagtgtgagcaggctgagcggctcggagcagtggacgaatctctgagtgaggagacccagaaggctgttcttcagtggacgaaacacgatgattcttcagacagcttctgtgaagttgatgacatacagtcccccgatgctgagtatgtggatttactccttaaccctgagcgctacacaggctacaaggggccggacgcttggaggatatggagtgtcatctatgaagaaaactgctttaagccacagacaattcaaaggcctttggcttcggggcaaggaaaacataaagagaacacattttacagctggctagaaggcctctgtgtagaaaagagagcattctacaggcttatatctggcctacacgcaagcatcaatgtacatttgagtgcaaggtatcttttacaagataattggctggaaaagaaatggggtcataatgtcacagagtttcagcagcgctttgatggggttttgacagaaggagaaggccccaggaggctgaagaacctgtactttctttacctgatagagttaagggctctctctaaagtgcttccgtttttcgagcgcccagattttcagctcttcactggaaataaagttcaggatgtggaaaacaaagagttacttctggagattcttcatgaagtcaagtcatttcctttgcattttgatgagaattctttttttgcgggggataaaaacgaagcacataagctaaaggaggacttccgcctacactttagaaacatctcgaggatcatggactgcgtcggctgcttcaagtgccgcctgtggggcaagcttcagactcagggtctgggcactgctctgaagatcttgttttctgaaaaactgatcgcaaatatgcccgaaagcggacccagttatgaattccagctaaccagacaagaaatagtgtcgttgttcaatgcattcggaaggatttccacaagtgtgagagaattagagaacttcagacacttgttacagaatgttcactgaggagggcggctggaacctgcttgtttctgcacaggggagtccagagggcagaatgtctgagcacggtgattgcagtgaccgtcctgagccaaacgttcatatcaagctgcctttgtcaaaggagagatacattgttttaagtaaatgacatttttaaacattgtgttcatgtttaatattattgtgaataaaagtagtattttggtaatgtacaaattttaatactaagcaaaagtaaggtcattaaattgccctatgatggggttggggatttagctcagtggtagagctcttgcctaggaagcgcaaggccctgggttcggtccccagctccgaaaaaaaagaaccccccccccaaaaaaaattgcccccataaaaagggtaggtgaatcctgccccaggctctccacctaaatttttttttgaaaacttttttcccccaagg

SEQ ID NO: 4 - DNA, 4121 bp, Rattus norvegicus
  misc_feature (16)..(17): n is any nucleotide
rtttttttttcctttnnaaanggnnaaagnnttccccccnccttccttcnanttaaaaatttggnancccaaaangcttnggggggcnnngggnncccntnggggnttggggagttncnccnggnganntttncaagnaanttaaanattttttcacccaatcnccnttttggggaaaagccttgccttcacctttccaaagccaacccgttttcaaaggcttcaggtacccccagttggggagaaggggcctttctggccaacccttgctggcaaacgatttggttcctgggaagatgatgttaagctaattcattctgccaaagccaaaatagtgtaacaagaacagcctggtaccggcttgtttatcccaaatcttcttctgcaagtggaccatctgctagcatcaatagtagcagtgtttcagcaggaagctacatgctgttcccaaagggatggcaatgcctctgtcaaggaaagacccaacttcaaatgctgccgatgggcctttgcttaaagcctcagtgtccagccctgtgaaagcatcttcttcccctgtgagatccgctccattcatcactagaaactgtgaggtgcagagtcctgagctacttcacaaaactgttagtcctctgaaaacagaggtgttgaaaccatgtgagaagccaactttatcccaggcacttcagcccaaagagggagctaacaaggaagtttgtctacagtcacagtccaaggacaaacttgcaacaccaggaggaagaggaattaagcctttcctggaacgctttggagagcgttgtcaagaacacagtaaagaaagtccaacttgcagagcatttcatagaaccccaaatatcactccaaatacaaaagctatccaggaaagattattcaagcaaaacacgtgtttcatctactaccccaatttagcacagcagctcaaacaggagcgtgaaaaggaactggcgtgtctccgtggccgatttgacaagggcagtctctggagtgcagagaaggatgaaaagtcaagaagcaaacagctagaaaccaacaggaagttcactgtcagaactctcccctcaagaaacaccaaattgtctcaaggcaccccgtcgacctctgtgtcagataaagtggctgagactccaaccgcagtgaagatttctggtacagagcctgcaggttccactgaaagcgaaatgacaaagtccagccctttgaaaataacattgtttttagaagaggagaagtccttaaaagtagcatcagacccggaggttgagcagaagactgaagcagtgcatgaagtagagatgagtgtggacgatgaggatatcaacagctccaagtcattaacgacatcttcagtganttccctagnggaanggggaactggacngtggaaaaganccaaggaggagatggaccaagtggggaacggaaagcagcgaggngcaggaagatgtgcngaatatctcctcaatntcttnacangntcccgctggctcagacggttcggcgtggtgaatctacagaatgtaatttcttcacctgagttggaattgagagactatagcctgagtgctccaagtcccaaaccaggaaaattccaaagaactcgtgtcccccgagcagaatctggtgacagcctcagttctgaggaccgggaccttctttacagcattgatgcatataggtctcaaagattcaaagaaacagaacgcccttccataaagcaagtgattgttcgaaaggaagatgttacttcaaaattgagtgaaaagaatggtgtcttttctggtcaagttaatatcaaacaaaaaatgcaggaactcaataatgacataaatttgcagcagacagtgatctatcaggccagccaggctctcaactgctgtgttgatgaagagcacgggaaaggatccctggaagaagctgaggcagaaaggctctttctgantgcaactgagaaaagagcacttctgattgacgaactgaataagctgaagagtgaaggacctcagaggagaaacaagaccgctgtcgcatcccagagtggatttgccccatgtaaagggtcagtcaccttgtcagagatctgcctgcctctgaaggcagagtttgtatgcagcaccgcgcaaaagccagagtcatcgaattactactacttaattatgctaaaagctggggctgagcagatggtggccaccccattagcaagtactgcaactctcttagtggtgatgnccctgacattccccaccacgttaccccngaangatgtttccaatgactttgaaataaatgttgaagtttacagcttggtacaaaagaaagattccctcaggcctgagaagaagaagaaggcgtccaagtttaaggctattactccaaagagactcctcacatctataacttcaaaaagcagccttcatgcttcagttatggccagtccaggaggtctcagtgctgtgcgcaccagcaactttaccctagttggatctcacacactctccttatcttctgttggagacactaagtttgctttggacaaggtaccttttttgtctccgttggaaggtcacatctgtttaaaaataagctgtcaagtgaattcagctgttgaggaaaagggtttccttaccatatttgaagatgttagtggctttggtgcctggcaccgaagatggtgtgttctctctggcaactgtatctcttactggacttacccagatgatgagaggcgaaagaatcccataggaaggataaatctggccaattgtatcagtcatcagatagaaccagccaacagagaattttgtgcaagacgcaacactctggaattgattactgtccgaccacaaagagaagacgatcgagaaactcttgtcagccatgtagagacacactctgtgtcacccaagaactggctctctgcagatactaaagaagagcgggatctctggatgcagaaactcaaccaggtcattgttgatattcgcctctggcagcctgatgcatgctacaagcctgttgggaagccttaagccgaggagcttctgcaccgtgagagactttgctagctgtgtcttcttaagaagacagttagaagcagcagatttgcaggttgtattctatgctttaaatataaaagggtatgtgcaaatattcactacatattgtgcagtatttatatcttttctatgtaaaacttcacccagtttgtcttgcattcgtacatgtttgacagtcaaatactaacaatattcatgagaattgatatccatgctaaatataacattaagagtcttgttttatagaaacctcactagccagttattcatgacaaaaactattataatcaagttctgatttgtcctttggagctgtgggtttgaaggtattaaggtctcaaacagaaacatttcaggacatgtttagtaaagagatgagaaaaggcagcaaacactagtttaagctgctcagagctgctttccgcagagctgtgggcaggacaccgtaacatttgggcctgcatagtctatgctgaagggttaagagtcacacagctagtgctcactctgaccctacgtgtgcagtgtggggcaccttctcacagtgctcaggctttacttaaacagctatttttcatgtagttgaggatcctcattaacatgttcagccttttctcttataacaagagcaaatgtaaattggaaaaacacatacataaggaatttctaccaagctgctgtgactactcctttgcttcccagagttcttgtctcgttttcctttcatgttgatctaaaacactttacaaatctgttttgagatcactgaaaaatatataaagctatgcattccctttaaagcccaatgccttcttgcaatttaaaaatattacaatgcatggctgcagtttttaaatagtctgtgtttctcctctgactgtcagtttattgatggtttcatttataaaacactaaattctatcacttgccattatatttcttactccatttaaatgtgggttttcttatgtatattataaaagtattttatgactcctacataaataaataatgtggaattgtcnaaancaaaaaaaaaaaaaa

SEQ ID NO: 5 - DNA, 2059 bp, Rattus norvegicus
acaaaccaccaaaccaccaaacctgtttactcagattcatggattgttcacatatgttttaaccactcaccccacctcacagaggtgaccgaacccaggacttcagtcatgctgggctagccctgcatccatgagctgtgtgccctcaggcccttgcttaagctcctacgtagacgtagatgtcctgtttttatttaaggatttgaaaaccagtcatgggcaccatgatttaacacaaaatacttcagtgtgatggtctaatttcctgaaaataattgtttgttcttctttcaaggaaaaaccaaaccttatgaatccgagccgaactattataagccttaaaataaggagccgcccgccccacatcccagtcacccagtgtttgagtttggttgccctttctcacctgtgtaatcacagggtatacaattcatgtttcttatgcatgaaattaattttctttccctctgtggagtggggctatattttagacaggtttttattcgtggaagctcttcactgagagcaatatttgaagtggcttaagaatttacgtcacagcatttataaatgatatacctcaaagttatgctcctttgatgtcatataatgtcttgagcagttaggacaggttgagatgtgacataagaaaaagcaggatatgtatgtaatggataggaatgtcactttacactgttgtgtattttctctgtccctaagacttggtgtagtgccaagcatacagttggtatctaatttttgttgatggaaagtgtatggatttagtataccttaagtgaatggtgtagcttgtgtaacaatgtaccctatctccccttccctctcactttttctttcaaatcgcataataaacccacagattagatcagctttctgggcggcgacttcgaaaagtactaaatgatcaccgcacagaagccagccctttgaaaccctcactgctttcacttgcgttctcccacttgactgtccctgtgtcctctgtctctccaaggaaggtctaaactcctacgtctttcgttaacaagcagtttaatttttaagaaatcttaacttttcctgtgcttgacacaattgacaatccctttcttcaagccccaccactctgcgtccttgtatctggcttgctcctgggtctcttccttctggtctcttcatgtaaccgaaatattaattccccagacttttctttcttgctctaagtcactggaccatactcttgtgtaatttccatgcagtcatcttatcttagcttctgttttcctgctgcggtcacttggctacctgttgccacgtcttcaaggactcacttcgtttgcgctcctcacttggttagtttcagaacattacactgttcaaggttctccagttcgctcttctgtcttctgcctgactatcggtgtctacgttctgctgcttctactccaacatttctatcactgtctttcaatttttattacagttactcaaaggatttcctgtgtttattttcccatctctgttggcccagattaccgaattgggctttctagaagcattcagcctcatccctgctacaggcagttttaggagctttttggtgagagtctctgcttggtatctaagaccctcctcttgtgtttgccactctgctctgataagagtgttaaagagttttccagaagtccagagttgtagccctccagaccttcgtagacaccatatttgcatggagagccctaggcttcttctgggaaactccatgcgttcttgagactctgtgacattaattaccctggcccttcctttggtcaccattatagttgcaacctacctctattgaatcacttattgtactgtatattttattttttaaagtgtcctttactagaatgtgagctcctcaggggcaggcaaagaaacttcattcatttggcatctctatagcataatgtttggtatatgagcatttaataaatgttgaataaattgcttcacatgacagctgttcctcatggcgggcgtcttcactgcctttgttgcaaaacgggggggaaaa

SEQ ID NO: 6 - DNA, 1987 bp, Rattus norvegicus
ctcgagagctccgccatggccgctctcacccgggacccccagttccagaagctgcagcaatggtaccgcgagcaccgctccgagctgaacctgcgccgcctcttcgatgccaacaaggaccgcttcaaccacttcagcttgaccctcaacaccaaccatgggcatatcctggtggattactccaagaacctggtgacggaggacgtgatgcggatgctggtggacttggccaagtccaggggcgtggaggccgcccgggagcggatgttcaatggtgagaagatcaactacaccgagggtcgagccgtgctgcacgtggctctgcggaaccggtcaaacacacccatcctggtagacggcaaggatgtgatgccagaggtcaacaaggttctggacaagatgaagtctttctgccagcgtgtccggagcggtgactggaaggggtacacaggcaagaccatcacggacgtcatcaacattggcattgtcggctccgacctgggacccctcatggtgactgaagcccttaagccatactcttcaggaggtccccgcgtctggtatgtctccaacattgatggaactcacattgccaaaaccctggcccagctgaacccggagtcctccctgttcatcattgcctccaagacctttactacccaggagaccatcacgaatgcagagacggcgaaggagtggtttctccaggcggccaaggatccttctgcagtggcgaagcactttgttgccctgtctactaacacaaccaaagtgaaggagtttggaattgaccctcaaaacatgttcgagttctgggattgggtgggaggacgctactcgctgtggtcggccatcggactctccattgccctgcacgtgggttttgacaacttcgagcagctgctctcgggggctcactggatggaccagcacttccgcacgacgcccctggagaagaacgcccccgtcttgctggccctgctgggtatctggtacatcaactgctttgggtgtgagacacacgccatgctgccctatgaccagtacctgcaccgctttgctgcgtacttccagcagggcgacatggagtccaatgggaaatacatcaccaaatctggaacccgtgtggaccaccagacaggccccattgtgtggggggagccagggaccaatggccagcatgctttttaccagctcatccaccaaggcaccaagatgataccctgtgacttcctcatcccggtccagacccagcaccccatacggaagggtctgcatcacaagatcctcctggccaacttcttggcccagacagaggccctgatgaggggaaaatcgacggaggaggcccgaaaggagctccaggctgcgggcaagagtccagaggaccttgagaggctgctgccacataaggtctttgaaggaaatcgcccaaccaactctattgtgttcaccaagctcacaccattcatgcttggagccttggtcgccatgtatgagcacaagatcttcgttcagggcatcatctgggacatcaacagctttgaccagtggggagtggagctgggaaagcagctggctaagaaaatagagcctgagcttgatggcagtgctcaagtgacctctcacgacgcttctaccaatgggctcatcaacttcatcaagcagcagcgcgaggccagagtccaataaactcgtgctcatctgcagcctcctctgtgactcccctttctcttctcgtccctcctccccggagccggcactgcatgttcctggacaccacccagagcaccctctggttgtgggcttggaccacgagcccttagcagggaaggctggtctcccccagcctaacccccagcccctccatgtctatgctccctctgtgttagaattggctgaagtgtttttgtgcagctgacttttctgacccatgttcacgttgttcacatcccatgtagaaaaacaaagatgccacggaggaggt

SEQ ID NO: 7 - protein, 464 aa, Rattus norvegicus
MGRGWGLLVGLLGVVWLLRSGQGEEQQQETAAQRCFCQVSGYLDDCTCDVETIDKFNNYRLFPRLQKLLESDYFRYYKVNLRKPCPFWNDINQCGRRDCAVKPCHSDEVPDGIKSASYKYSKEANLLEECEPAERLGAVDESLSEETQKAVLQWTKHDDSSDSFCEVDDIQSPDAEYVDLLLNPERYTGYKGPDAWRIWSVIYEENCFKPQTFQRPLASGQGKHKENTFYSWLEGLCVEKRAFYRLISGLHASINVHLSARYLLQDNWLEKKWGHNVTEFQQRFDGVLTEGEGPRRLKNLYFLYLIELRALSKVLPFFERPDFQLFTGNKVQDVENKELLLEILHEVKSFPLHFDENSFFAGDKNEAHKLKEDFRLHFRNISRIMDCVGCFKCRLWGKLQTQGLGTALKILFSEKLIANMPESGPSYEFQLTRQEIVSLFNAFGRISTSVRELENFRHLLQNVH

SEQ ID NO: 8 - protein, 558 aa, Rattus norvegicus [sequence omitted]

SEQ ID NO: 9 - protein, 229 aa, Rattus norvegicus
MPSLWDRFSSSSSSSSSSRTPAADRPPRSAWGSAAREEGLDRCASLESSDCESLDSSNSGFGPEEDSSYLDGVSLPDFELLSDPEDEHLCANLMQLLQESLSQARLGSRRPARLLMPSQLLSQVGKELLRLAYSEPCGLRGALLDVCVEQGKSCHSVAQLALDPSLVPTFQLTLVLRLDSRLWPKIQGLLSSANSSLVPGYSQSLTLSTGFRVIKKKLYSSEQLLIEEC

SEQ ID NO: 10 - protein, 232 aa, Homo sapiens
MPSLWDRFSSSSTSSSPSSLPRTPTPDRPPRSAWGSATREEGFDRSTSLESSDCESLDSSNSGFGPEEDTAYLDGVSLPDFELLSDPEDEHLCANLMQLLQESLAQARLGSRRPARLLMPSQLVSQVGKELLRLAYSEPCGLRGALLDVCVEQGKSCHSVGQLALDPSLVPTFQLTLVLRLDSRLWPKIQGLFSSANSPFLPGFSQSLTLSTGFRVIKKKLYSSEQLLIEEC

SEQ ID NO: 11 - protein, 864 aa, Rattus norvegicus
  misc_feature (307)..(307): Xaa is unknown
MAMPLSRKDPTSNAADGPLLKASVSSPVKASSSPVRSAPFITRNCEVQSPELLHKTVSPLKTEVLKPCEKPTLSQALQPKEGANKEVCLQSQSKDKLATPGGRGIKPFLERFGERCQEHSKESPTCRAFHRTPNITPNTKAIQERLFKQNTCFIYYPNLAQQLKQEREKELACLRGRFDKGSLWSAEKDEKSRSKQLETNRKFTVRTLPSRNTKLSQGTPSTSVSDKVAETPTAVKISGTEPAGSTESEMTKSSPLKITLFLEEEKSLKVASDPEVEQKTEAVHEVEMSVDDEDINSSKSLTTSSVXSLXEXGTGXWKRXKEEMDQVGNGKQRGAGRCAEYLLNXXTXSRWLRRFGVVNLQNVISSPELELRDYSLSAPSPKPGKFQRTRVPRAESGDSLSSEDRDLLYSIDAYRSQRFKETERPSIKQVIVRKEDVTSKLSEKNGVFSGQVNIKQKMQELNNDINLQQTVIYQASQALNCCVDEEHGKGSLEEAEAERLFLXATEKRALLIDELNKLKSEGPQRRNKTAVASQSGFAPCKGSVTLSEICLPLKAEFVCSTAQKPESSNYYYLIMLKAGAEQMVATPLASTATLLVVMXLTFPTTLPXXDVSNDFEINVEVYSLVQKKDSLRPEKKKKASKFKAITPKRLLTSITSKSSLHASVMASPGGLSAVRTSNFTLVGSHTLSLSSVGDTKFALDKVPFLSPLEGHICLKISCQVNSAVEEKGFLTIFEDVSGFGAWHRRWCVLSGNCISYWTYPDDERRKNPIGRINLANCISHQIEPANREFCARRNTLELITVRPQREDDRETLVSHVETHSVSPKNWLSADTKEERDLWMQKLNQVIVDIRLWQPDACYKPVGKP

(19) United States
(12) Patent Application Publication
(10) Pub. No.: US 2003/0104973 A1
(43) Pub. Date: Jun. 5, 2003

(54) Hypoxia-regulated genes

(75) Inventors: Paz Einat, Nes Ziona (IL); Rami Skaliter, Nes Ziona (IL)

Correspondence Address: BROWDY AND NEIMARK, P.L.L.C., 624 NINTH STREET, NW, SUITE 300, WASHINGTON, DC, 20001-5303, US

(73) Assignee: Quark Biotech, Inc., Pleasanton, CA

(21) Appl. No.: 10/091,333

(22) Filed: Mar. 6, 2002

Related U.S. Application Data
(63) Continuation-in-part of application No. 09/604,978, filed on Jun. 28, 2000, now Pat. No. 6,455,674.
Application No. 09/604,978 is a division of application No. 09/138,112, filed on Aug. 21, 1998, now abandoned.
(60) Provisional application No. 60/056,453, filed on Aug. 21, 1997.

(51) Int. Cl. A61K 31/00; C07H 21/04; C07K 14/47; C07K 16/40

(52) U.S. Cl. 514/1; 530/350; 530/388.1; 536/23.1

(57) ABSTRACT

According to the present invention, purified, isolated and cloned nucleic acid polynucleotide encoding hypoxia-regulating genes and the proteins thereof and antibodies directed against the proteins which have sequences as set forth in SEQ ID NO:1, SEQ ID NO:2, SEQ ID NO:3, SEQ ID NO:4, SEQ ID NO:5 and SEQ ID NO:6 are provided. The present invention further provides transgenic animals and cell lines as well as knock-out organisms of these sequences. The present invention further provides methods of regulating angiogenesis or apoptosis or regulating response to ischemic or hypoxic conditions in a patient in need of such treatment. The present invention also provides a method of diagnosing the presence of ischemia in a patient including the steps of analyzing a bodily fluid or tissue sample from the patient for the presence, or gene product, of at least one expressed gene (up-regulated) as set forth in the group comprising SEQ ID NO:2; SEQ ID NO:3; SEQ ID NO:4; SEQ ID NO:5; and SEQ ID NO:6 and where ischemia is determined if the up-regulated gene or gene product is ascertained.

### CROSS-REFERENCE TO RELATED APPLICATIONS

This application is a continuation-in-part of application Ser. No. 09/604,978, filed Jun. 28, 2000, the entire contents of which are hereby incorporated herein by reference. Said application Ser. No. 09/604,978 is a divisional of application Ser. No. 09/138,112, filed Aug. 21, 1998, now abandoned, which claims priority from U.S. Ser. No. 60/056,453, filed Aug. 21, 1997.

### BACKGROUND OF THE INVENTION

1. Field of the Invention

Identification of genes that are differentially expressed in hypoxia and use of the genes and gene products for diagnosis and therapeutic intervention.

2. Description of Related Art

The level of tissue oxygenation plays an important role in normal development as well as in pathologic processes such as ischemia. Tissue oxygenation plays a significant regulatory role in both apoptosis and in angiogenesis (Bouck et al, 1996; Bunn et al, 1996; Dor et al, 1997; Carmeliet et al, 1998). Apoptosis (see Duke et al, 1996 for review) and growth arrest occur when cell growth and viability are reduced due to oxygen deprivation (hypoxia). Angiogenesis (i.e. blood vessel growth, vascularization), is stimulated when hypooxygenated cells secrete factors that stimulate proliferation and migration of endothelial cells in an attempt to restore oxygen homeostasis (for review see Hanahan et al, 1996).

Ischemic disease pathologies involve a decrease in the blood supply to a bodily organ, tissue or body part generally caused by constriction or obstruction of the blood vessels as for example retinopathy, acute renal failure, myocardial infarction and stroke. Therefore apoptosis and angiogenesis as induced by the ischemic condition are also involved in these disease states. Neoangiogenesis is seen in some forms of retinopathy and in tumor growth. It is recognized that angiogenesis is necessary for tumor growth and that retardation of angiogenesis would be a useful tool in controlling malignancy and retinopathies. Further, it would be useful to induce tumorigenic cells to undergo apoptosis (i.e., programmed cell death).

However, these processes are complex cascades of events controlled by many different genes reacting to the various stresses such as hypoxia. Expression of different genes reacting to the hypoxic stress can trigger not only apoptosis or angiogenesis but also both. In cancer it has been observed that apoptosis- and angiogenesis-related genes are therapeutic targets. However, hypoxia itself plays a critical role in the selection of mutations that contribute to more severe tumorigenic phenotypes (Graeber et al, 1996). Therefore identifying candidate genes and gene products that can be utilized therapeutically not only in cancer and ischemia and that may either induce apoptosis or angiogenesis or to retard the processes is needed. It would be useful to identify genes that have direct causal relationships between a disease and its related pathologies and an up- or down-regulator (responder) gene.

### SUMMARY OF THE INVENTION

According to the present invention, purified, isolated and cloned nucleic acid sequences encoding hypoxia-responding genes which have sequences as set forth in the group comprising SEQ ID NO:1, SEQ ID NO:2, SEQ ID NO:3, SEQ ID NO:4 and SEQ ID NO:5 or a complementary or allelic variation sequence thereof and human homologs as needed thereto. The present invention further provides proteins as encoded by the nucleic acid sequences as set forth in SEQ ID NO:1, SEQ ID NO:2, SEQ ID NO:3, SEQ ID NO:4, SEQ ID NO:5 and SEQ ID NO:6 with SEQ ID NOs:7-11 being exemplars of the proteins. The present invention further provides antibodies directed against the proteins as encoded by the nucleic acid sequences as set forth in SEQ ID NO:1, SEQ ID NO:2, SEQ ID NO:3, SEQ ID NO:4, SEQ ID NO:5 and SEQ ID NO:6 including SEQ ID NOs:7-11.

The present invention further provides transgenic animals and cell lines carrying at least one of the expressible nucleic acid sequences as set forth in SEQ ID NO:1, SEQ ID NO:2, SEQ ID NO:3, SEQ ID NO:4, SEQ ID NO:5 and SEQ ID NO:6. The present invention further provides knock-out eucaryotic organisms in which at least one of the nucleic acid sequences as set forth in SEQ ID NO:1, SEQ ID NO:2, SEQ ID NO:3, SEQ ID NO:4, SEQ ID NO:5 and SEQ ID NO:6 is knocked-out.

The present invention provides a method of regulating angiogenesis in a patient in need of such treatment by administering to a patient a therapeutically effective amount of an antagonist of at least one protein as encoded by the nucleic acid sequences as set forth in SEQ ID NO:2, SEQ ID NO:3, SEQ ID NO:4, SEQ ID NO:5 and SEQ ID NO:6. Alternatively, the present invention provides a method of regulating angiogenesis in a patient in need of such treatment by administering to a patient a therapeutically effective amount of at least one antisense oligonucleotide against the nucleic acid sequences as set forth in SEQ ID NO:2, SEQ ID NO:3, SEQ ID NO:4, SEQ ID NO:5 and SEQ ID NO:6 or a dominant negative peptide directed against the sequences or their proteins.

The present invention further provides a method of regulating angiogenesis or apoptosis in a patient in need of such treatment by administering to a patient a therapeutically effective amount of a protein encoded by SEQ ID NOs:2-6 or the protein sequences as set forth in SEQ ID NOs:7-8,10-11 as active ingredients in a pharmaceutically acceptable carrier.

The present invention provides a method of providing an apoptotic regulating gene by administering directly to a patient in need of such therapy, utilizing gene therapy, an expressible vector comprising expression control sequences operably linked to one of the sequences set forth in the group comprising SEQ ID NO:2; SEQ ID NO:3; SEQ ID NO:4; SEQ ID NO:5 and SEQ ID NO:6 (human homolog).

The present invention also provides a method of providing an angiogenesis-regulating gene utilizing gene therapy by administering directly to a patient in need of such therapy an expressible vector comprising expression control sequences operably linked to one of the sequences set forth in the group comprising SEQ ID NO:2; SEQ ID NO:3; SEQ ID NO:4; SEQ ID NO:5 and SEQ ID NO:6.

The present invention provides a method of regulating response to ischemic or hypoxic conditions in a patient in need of such treatment by administering to a patient a therapeutically effective amount of an antisense oligonucleotide directed against at least one of the sequences set forth in the group comprising SEQ ID NO:2; SEQ ID NO:3; SEQ ID NO:4; SEQ ID NO:5; and SEQ ID NO:6. The present invention further provides a method of providing an hypoxia regulating gene utilizing gene therapy by administering directly to a patient in need of such therapy an expressible vector comprising expression control sequences operably linked to one of the sequences set forth in the group comprising SEQ ID NO:2; SEQ ID NO:3; SEQ ID NO:4; SEQ ID NO:5 and SEQ ID NO:6.

The present invention also provides a method of diagnosing the presence of ischemia in a patient including the steps of analyzing a bodily fluid or tissue sample from the patient for the presence or gene product of at least one expressed gene (up-regulated) as set forth in the group comprising SEQ ID NO:2; SEQ ID NO:3; SEQ ID NO:4; SEQ ID NO:5; and SEQ ID NO:6 and where ischemia is determined if the up-regulated gene or gene product is ascertained.

### DESCRIPTION OF THE DRAWINGS

Other advantages of the present invention will be readily appreciated as the same becomes better understood by reference to the following detailed description when considered in connection with the accompanying drawings wherein:

FIG. 1 is a computer scan showing in-vitro translation of full length cDNA clones of RTP801 (SEQ ID NO:1). cDNA clones were translated in-vitro using a coupled transcription translation kit (Promega). Translation products were separated on acrylamide gel and exposed to X-ray film. Two clones, marked with arrows, gave the expected protein size of approximately 30 KD. This confirms the sequence analysis of the putative reading frame.

FIG. 2 is a computer scan showing RTP801 (SEQ ID NO:1) Northern blot analysis. RNA was extracted from Rat C6 glioma cells which were exposed to hypoxia for 0, 4, or 16 hours. PolyA+ selected mRNA (2 μg) from each sample were separated on denaturing agarose gels, blotted onto Nytran membranes and hybridized with RTP241 probe. One band of 1.8 Kb is observed showing a marked induction after hypoxia.

FIG. 3 is a computer scan showing RTP779 (SEQ ID NO:2) Northern blot analysis. RNA was extracted from Rat C6 glioma cells which were exposed to hypoxia for 0, 4, or 16 hours. PolyA+ selected mRNA (2 μg) from each sample was separated on denaturing agarose gels, blotted onto Nytran membranes and hybridized with RTP779 probe. One band of 1.8 Kb is observed showing extreme differential expression.

FIG. 4 is a computer scan showing RTP241 (SEQ ID NO:3) Northern blot analysis. RNA was extracted from Rat C6 glioma cells which were exposed to hypoxia for 0, 4, or 16 hours. PolyA+ selected mRNA (2 μg) from each sample were separated on denaturing agarose gels, blotted onto Nytran membranes and hybridized with RTP241 probe. Two bands of 1.8 Kb and 4 Kb are observed; both show good differential expression.

FIG. 5 is a computer scan showing RTP359 (SEQ ID NO:5) Northern blot analysis. RNA was extracted from Rat C6 glioma cells which were exposed to hypoxia for 0, 4, or 16 hours. PolyA+ selected mRNA (2 μg) from each sample was separated on denaturing agarose gels, blotted onto Nytran membranes and hybridized with RTP359 probe. One band of 4.5 Kb is observed showing good differential expression.

FIGS. 6A-D show the transcriptional regulation of RTP801.

FIG. 6A is a Northern blot analysis of RTP801 transcription in wild type mouse ES cells (ES+/+) and in HIF-1α null mouse ES cells (ES−/−) cultured under normoxic (N) or hypoxic conditions (H) for 16 hours. 15 μg of total RNA were loaded per slot.

FIG. 6B shows the nucleotide sequences of immediate upstream genomic regions of mouse and human RTP801 orthologs. The initiation ATG codon is shown in bold and the position of T is counted as +1. The TATA box is gray-shaded. Putative hypoxia response elements (HRE) are shown with white letters in black boxes. A putative Egr-1 binding site is marked with a dashed line.

FIG. 6C presents electrophoretic mobility shift assays and supershift analysis of the mouse RTP801 promoter region. All the binding reactions except for those loaded in lanes 2, 4 and 5 were performed with nuclear extracts prepared from wild type ES cells cultured under hypoxic conditions for 16 hours. The reaction mixture loaded in lane 2 contains nuclear extract prepared from wild type ES cells cultured in normoxia, whereas reaction mixtures loaded in lanes 4 and 5 contain nuclear extracts from HIF-1α −/− ES cells maintained in normoxic and hypoxic conditions, respectively. Lane 1: ³²P-TR-HRE oligonucleotide, lanes 2-5: ³²P-RTP801-HRE oligonucleotide, lane 6: ³²P-RTP801-HRE oligonucleotide and an excess of non-labeled RTP801-HRE oligonucleotide, lane 7: ³²P-RTP801-HRE oligonucleotide and an excess of non-labeled TR-HRE oligonucleotide, lane 8: ³²P-RTP801-HRE oligonucleotide and anti-HIF-1α antibodies, lane 9: ³²P-RTP801-HRE oligonucleotide and anti-Flag antibodies; lane 10: ³²P-RTP801-MHRE oligonucleotide.

FIG. 6D is a Northern blot analysis demonstrating the p53-independence of hypoxic transactivation of RTP801. H1299 is a human lung carcinoma p53-negative cell line that was engineered to express the wild type p53 under the control of tetracycline-repressible promoter. The cells were cultured either in the absence (left panel) or in the presence (right panel) of tetracycline to induce (left) or to suppress (right) p53 expression, respectively. Both p53-positive and p53-negative H1299 cells were maintained either in normal (N) or in hypoxic conditions (H) or in the presence of doxorubicin (D). 15 μg of total RNA derived from each experiment were analyzed by Northern blot using the probes for human RTP801 and for Waf1 (as a positive control for p53-dependent transactivation).

FIG. 7 shows an assessment of cytotoxic effect of H₂O₂ treatment and ischemic treatment in control and RTP801-expressing differentiated PC12-Tet-Off cells. 1-parental PC12-Tet-Off clone; 2-PC12-vector (a clone of PC12-Tet-Off cells transfected with the empty pSHTet vector). 3-PC801-10.

FIG. 8 shows the inhibition of RTP801-induced cytotoxicity in differentiated PC12-Tet-Off cells by Boc-D (OMe)-FMK caspase inhibitor. PC12-Tet-Off clone; V-PC12-vector (a clone of PC12-Tet-Off cells transfected with the empty pSHTet vector); 10-PC801-10. I-Boc-D (OMe)-FMK caspase inhibitor.

FIG. 9 shows the inducible expression of RTP801 and sensitization of MCF7 and PC12 cells to serum deprivation. Left: 1-parental MCF7-Tet-Off clone; 2-MCF7-vector (a clone of MCF7-Tet-Off cells transfected with the empty pSH-Tet vector); 3-MCF801-8; 4-MCF7801-12. Right: 1-parental PC12-Tet-Off clone; 2-PC12-vector (a clone of PC12-Tet-Off cells transfected with the empty pSH-Tet vector); 3-PC801-10.

FIG. 10 relates to the liposomal delivery of RTP801 cDNA into mouse lungs. FIG. 10 shows a Northern blot analysis of RNA (15 μg per lane) extracted from lungs of mice injected with liposomes containing either pcDNA3 DNA (lanes 1-3) or pcDNA3-RTP801 (lanes 4-5). Position of the RTP801-specific band is indicated.

FIG. 11 is a Northern blot analysis of RNA extracted from cortex of RTP801 transgenic mice using SV40 transgene-specific probe. 11-RTP801 higher expresser transgenic line; 1-RTP801 lower expresser transgenic line.

FIG. 12 is an analysis of damage volume distribution in consecutive slices in five 801-11 transgenic mice in comparison with damage volume distribution in five wild type littermates.

FIG. 13 is a Western blot analysis, using RTP801 polyclonal antibody to detect the expression of the 801 gene under various conditions that induce oxidative stress in PC12 cells. N-normoxia; I-4-Ischemia for 4 hours; I-24-Ischemia for 24 hours; H-4-H₂O₂ for 4 hours; D-8-DFO for 8 hours; D-5-DFO for 5 hours; 10-T-Tet induced clone grown in the absence of tetracycline; 10+T-Tet induced clone grown in the presence of tetracycline; 293-HEK293 cells transiently transfected with RTP801 expression plasmid.

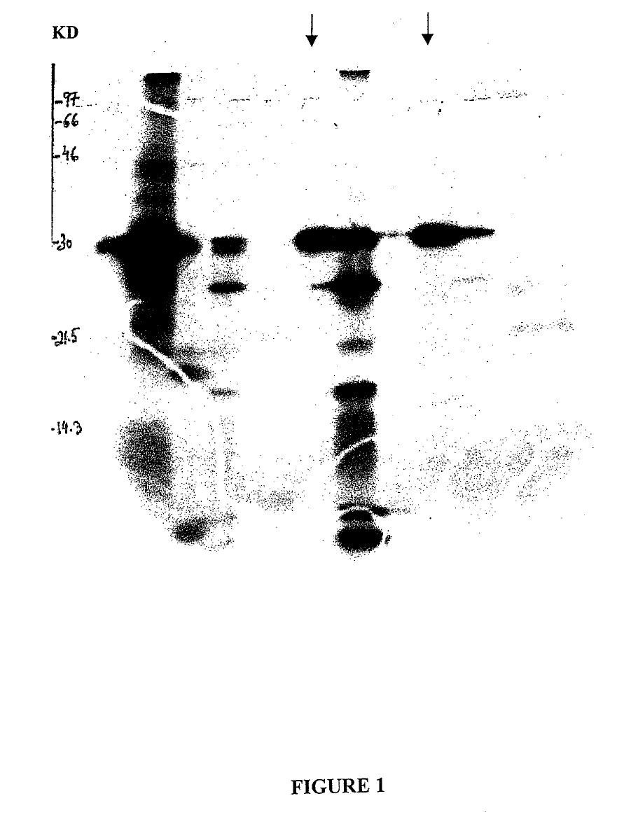

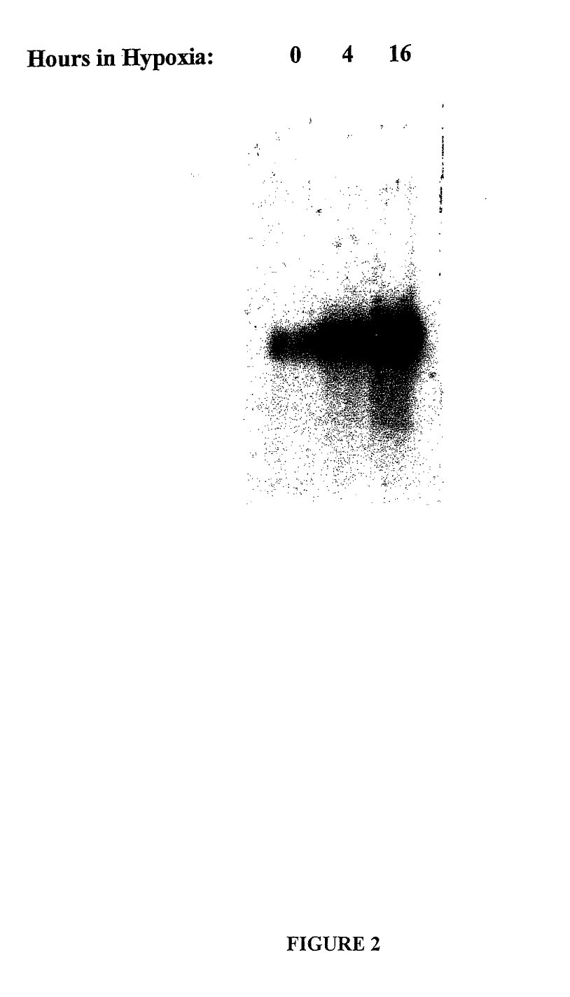

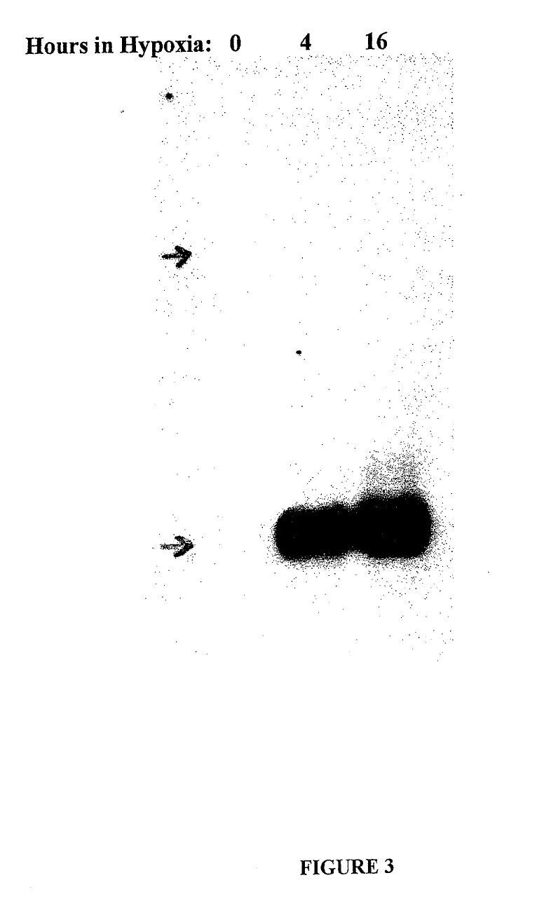

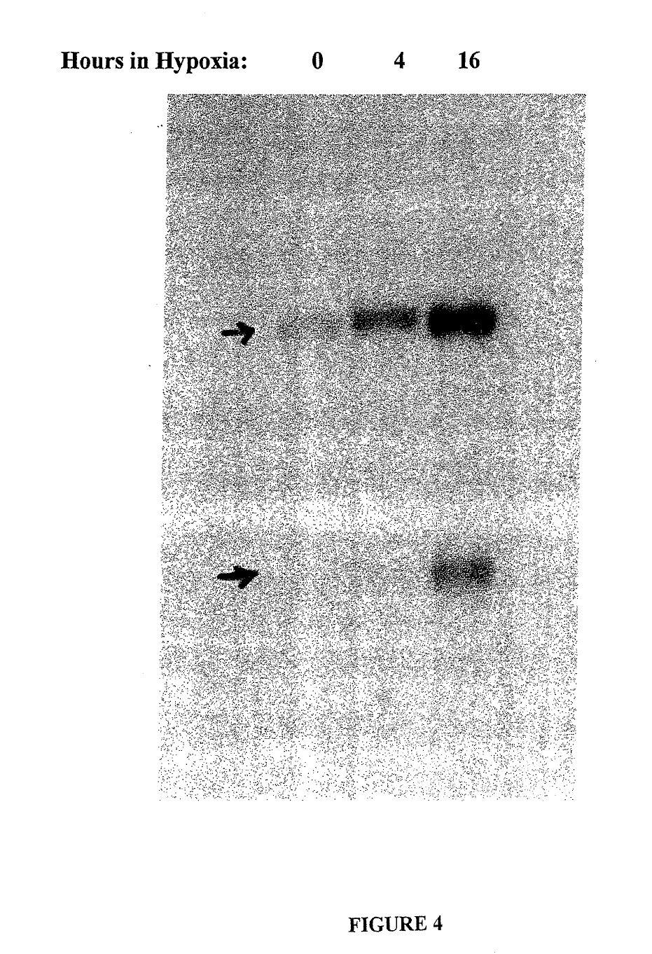

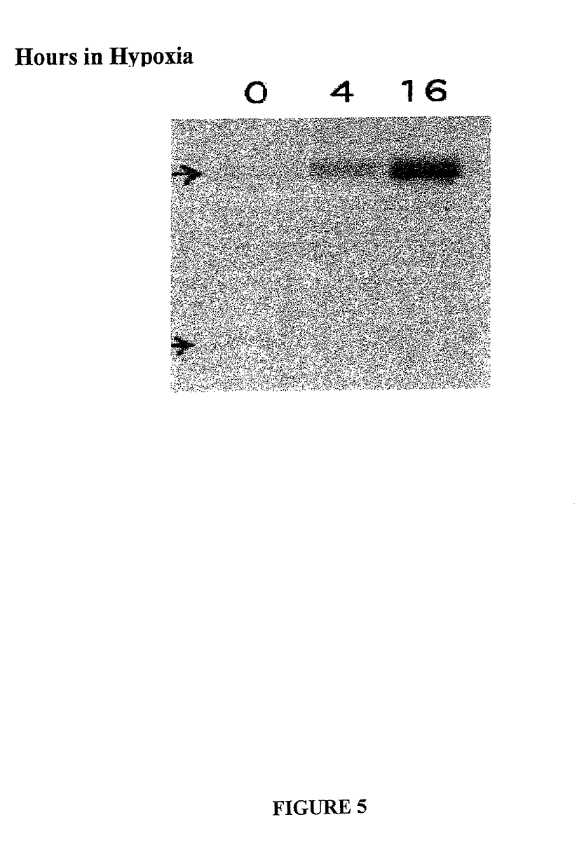

### DETAILED DESCRIPTION OF THE INVENTION

The present invention identifies candidate genes and gene products that can be utilized therapeutically and diagnostically in hypoxia and ischemia and that may regulate apoptosis or angiogenesis. By regulate or modulate or control is meant that the process is either induced or inhibited to the degree necessary to effect a change in the process and the associated disease state in the patient. Whether induction or inhibition is being contemplated will be apparent from the process and disease being treated and will be known to those skilled in the medical arts. The present invention identifies genes for gene therapy, diagnostics and therapeutics that have direct causal relationships between a disease and its related pathologies and up- or down-regulator (responder) genes. That is, the present invention is initiated by a physiological relationship between cause and effect.

The present invention provides purified, isolated and cloned nucleic acid polynucleotides (sequences) encoding genes which respond at least to hypoxic conditions by up-regulation of expression and which have sequences as set forth in the group comprising SEQ ID NO:1, SEQ ID NO:2, SEQ ID NO:3, SEQ ID NO:4 and SEQ ID NO:5 and their analogues and polymorphisms or a complementary or allelic variation sequence thereto. The present invention further provides SEQ ID NO:6 which is a known gene (neuroleukin) which also responds to the stress of hypoxia by being up-regulated. SEQ ID NO:6 is the human sequence for neuroleukin and has over 90% homology with the rat sequence. The human homolog is used where appropriate. Because of the high homology between the rat and human sequences the rat sequence can also be used for probes and the like as necessary.

The present invention further provides proteins and their analogues as encoded by the nucleic acid sequences as set forth in SEQ ID NO:1, SEQ ID NO:2, SEQ ID NO:3, SEQ ID NO:4, SEQ ID NO:5 with SEQ ID NOs:7 and 8 as well as SEQ ID NOs:9-11 being exemplars of the proteins. The present invention further provides a method of regulating angiogenesis or apoptosis in a patient in need of such treatment by administering to a patient a therapeutically effective amount of a protein encoded by SEQ ID NOs:2-6 or the protein sequences as set forth in SEQ ID-8,10-11 as active ingredients in a pharmaceutically acceptable carrier.

The proteins may be produced recombinantly (see generally Marshak et al, 1996) and analogues may be due to post-translational processing. The term “analogue” as used herein is defined as a nucleic acid sequence or protein which has some differences in its amino acid/nucleotide sequences as compared to the native sequence of SEQ ID NOs:1-8. Ordinarily, the analogue will be generally at least 70% homologous over any portion that is functionally relevant. In more preferred embodiments the homology will be at least 80% and can approach 95% homology to the protein/nucleotide sequence.

The amino acid or nucleotide sequence of an analogue may differ from that of the primary sequence when at least one residue is deleted, inserted or substituted, but the protein or nucleic acid molecule remains functional. Differences in glycosylation can provide protein analogues.

Functionally relevant refers to the biological property of the molecule and in this context means an in vivo effector or antigenic function or activity that is directly or indirectly performed by a naturally occurring protein or nucleic acid molecule. Effector functions include, but are not limited to, receptor binding, any enzymatic activity or enzyme modulatory activity, any carrier binding activity, any hormonal activity, any activity in promoting or inhibiting adhesion of cells to extracellular matrix or cell surface molecules, or any structural role as well as having the nucleic acid sequence encode functional protein and be expressible. The antigenic functions essentially mean the possession of an epitope or antigenic site that is capable of cross-reacting with antibodies raised against a naturally occurring protein. Biologically active analogues share an effector function of the native which may, but need not, in addition possess an antigenic function.

The present invention further provides antibodies directed against the proteins as encoded by the nucleic acid sequences as set forth in SEQ ID NO:1, SEQ ID NO:2, SEQ ID NO:3, SEQ ID NO:4, SEQ ID NO:5 and SEQ ID NO:6 which can be used in immunoassays and the like.

The antibodies may be monoclonal, polyclonal or recombinant. Conveniently, the antibodies may be prepared against the immunogen or portion thereof, for example, a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane (1988) and Borrebaeck (1992). Antibody fragments may also be prepared from the antibodies and include Fab, F(ab′)₂, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of splenic antibody producing cells. These cells are fused to a cell having immortality, such as a myeloma cell, to provide a fused cell hybrid that has immortality and secretes the required antibody. The cells are then cultured in bulk and the monoclonal antibodies harvested from the culture media for use.

For producing recombinant antibody (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone et al (1982).) The binding of antibodies to a solid support substrate is also well known in the art (see for a general discussion Harlow & Lane, 1988 and Borrebaeck, 1992). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

The present invention further provides transgenic animals and cell lines carrying at least one expressible nucleic acid sequence as set forth in SEQ ID NO:1, SEQ ID NO:2, SEQ ID NO:3, SEQ ID NO:4, SEQ ID NO:5 and SEQ ID NO:6. By expressible is meant the inclusion with the sequence of all regulatory elements necessary for the expression of the gene or by the placing of the gene in the target genome so that it is expressed. The present invention further provides knock-out eucaryotic organisms in which at least one nucleic acid sequence as set forth in SEQ ID NO:1, SEQ ID NO:2, SEQ ID NO:3, SEQ ID NO:4, SEQ ID NO:5 and SEQ ID NO:6 is knocked-out.

These transgenics and knock-outs are constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384,5,175,383, 4,736,866 as well as Burke and Olson (1991), Capecchi (1989), Davies et al (1992), Dickinson et al (1993), Duff and Lincoln (1995), Huxley et al (1991), Jakobovits et al (1993), Lamb et al (1993), Pearson and Choi (1993), Rothstein (1991), Schedl et al (1993), Strauss et al (1993). Further, patent applications WO 94/23049, WO 93/14200, WO 94/06908, WO 94/28123 also provide information.

More specifically, any techniques known in the art may be used to introduce the transgene expressibly into animals to produce the parental lines of animals. Such techniques include, but are not limited to, pronuclear microinjection (U.S. Pat. No. 4,873,191); retrovirus mediated gene transfer into germ lines (Van der Putten et al, 1985); gene targeting in embryonic stem cells (Thompson et al, 1989; Mansour, 1990 and U.S. Pat. No. 5,614,396); electroporation of embryos (Lo, 1983); and sperm-mediated gene transfer (Lavitrano et al, 1989). For a review of such techniques see Gordon (1989).

Further one parent strain, instead of carrying a direct human transgene, may have the homologous endogenous gene modified by gene targeting such that it approximates the transgene. That is, the endogenous gene has been “humanized” and/or mutated (Reaume et al, 1996). It should be noted that, if the animal and human sequence are essentially homologous, a “humanized” gene is not required. The transgenic parent can also carry an overexpressed sequence, either the non-mutant or a mutant sequence and humanized or not as required. The term transgene is therefore used to refer to all these possibilities.

Additionally, cells can be isolated from the offspring which carry a transgene from each transgenic parent and that are used to establish primary cell cultures or cell lines as is known in the art.

Where appropriate, a parent strain will be homozygous for the transgene. Additionally, where appropriate, the endogenous non-transgene in the genome that is homologous to the transgene will be non-expressive. By non-expressive is meant that the endogenous gene will not be expressed and that this non-expression is heritable in the offspring. For example, the endogenous homologous gene could be “knocked-out” by methods known in the art. Alternatively, the parental strain that receives one of the transgenes could carry a mutation at the endogenous homologous gene rendering it non-expressed.

The present invention provides a method of regulating angiogenesis in a patient in need of such treatment by administering to a patient a therapeutically effective amount of an antagonist of at least one protein as encoded by the nucleic acid sequences as set forth in SEQ ID NO:1, SEQ ID NO:2, SEQ ID NO:3, SEQ ID NO:4, SEQ ID NO:5 and SEQ ID NO:6. The antagonist is dosed and delivered in a pharmaceutically acceptable carrier as described hereinbelow. The term antagonist or antagonizing is used in its broadest sense. Antagonism can include any mechanism or treatment that results in inhibition, inactivation, blocking or reduction in gene activity or gene product. It should be noted that the inhibition of a gene or gene product may provide for an increase in a corresponding function that the gene or gene product was regulating. The antagonizing step can include blocking cellular receptors for the gene products of SEQ ID NOs:1-6 and can include antisense treatment as discussed hereinbelow.

The present invention further provides a method of regulating angiogenesis or apoptosis in a patient in need of such treatment by administering to a patient a therapeutically effective amount of a regulating agent for a protein selected from the group consisting of SEQ ID NOs:7-11 in a pharmaceutically acceptable carrier. The regulating agent is dosed and delivered in a pharmaceutically acceptable carrier as described hereinbelow. For example, a patient may be in need of inducing apoptosis in tumorigenic cells or angiogenesis in trauma situations where for example a limb must be reattached or in a transplant where revascularization is needed.

The present invention provides a method of regulating angiogenesis or apoptosis in a patient in need of such treatment by administering to a patient a therapeutically effective amount of at least one antisense oligonucleotide or dominant negative peptide (either as cDNA or peptide; Herskowitz, 1987) directed against the nucleic acid sequences as set forth in SEQ ID NO:1, SEQ ID NO:2, SEQ ID NO:3, SEQ ID NO:4, SEQ ID NO:5 and SEQ ID NO:6. The present invention also provides a method of regulating response to hypoxic conditions in a patient in need of such treatment by administering to a patient a therapeutically effective amount of an antisense oligonucleotide directed against at least one of the sequences set forth in the group comprising SEQ ID NO:1; SEQ ID NO:2; SEQ ID NO:3; SEQ ID NO:4; SEQ ID NO:5; and SEQ ID NO:6. The antisense oligonucleotide as the active ingredient in a pharmaceutical composition is dosed and delivered in a pharmaceutically acceptable carrier as discussed hereinbelow.

Preferably, the present invention discloses a method for the treatment of a subject in need of treatment for hypoxia or ischemia-related disease (such as stroke) comprising administering to said subject a therapeutically effective amount of an antagonist of a protein having a sequence as set forth in SEQ ID NO:10, or an analogue thereof.

The present invention also discloses the use of an antagonist of a protein having a sequence as set forth in SEQ ID NO:10, or an analogue thereof, in the treatment of a subject in need of treatment for hypoxia or ischemia-related disease (such as stroke) in an amount sufficient to effect an inhibition or inactivation of the protein so as to thereby treat the subject.

Many reviews have covered the main aspects of antisense (AS) technology and its enormous therapeutic potential (Wright and Anazodo, 1995). There are reviews on the chemical (Crooke, 1995; Uhlmann et al, 1990), cellular (Wagner, 1994) and therapeutic (Hanania, et al, 1995; Scanlon, et al, 1995; Gewirtz, 1993) aspects of this rapidly developing technology. Within a relatively short time, ample information has accumulated about the in vitro use of AS nucleotide sequences in cultured primary cells and cell lines as well as for in vivo administration of such nucleotide sequences for suppressing specific processes and changing body functions in a transient manner. Further, enough experience is now available in vitro and in vivo in animal models and human clinical trials to predict human efficacy.

Antisense intervention in the expression of specific genes can be achieved by the use of synthetic AS oligonucleotide sequences (for recent reports see Lefebvre-d'Hellencourt et al, 1995; Agrawal, 1996; Lev-Lehman et al, 1997). AS oligonucleotide sequences may be short sequences of DNA, typically 15-30 mer but may be as small as 7 mer (Wagner et al, 1996), designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996). In that case, RNase H will cleave the RNA component of the duplex and can potentially release the AS to further hybridize with additional molecules of the target RNA. An additional mode of action results from the interaction of AS with genomic DNA to form a triple helix that may be transcriptionally inactive.

The sequence target segment for the antisense oligonucleotide is selected such that the sequence exhibits suitable energy-related characteristics important for oligonucleotide duplex formation with their complementary templates, and shows a low potential for self-dimerization or self-complementation (Anazodo et al, 1996). For example, the computer program OLIGO (Primer Analysis Software, Version 3.4), can be used to determine antisense sequence melting temperature, free energy properties, and to estimate potential self-dimer formation and self-complementary properties. The program allows the determination of a qualitative estimation of these two parameters (potential self-dimer formation and self-complementary) and provides an indication of “no potential” or “some potential” or “essentially complete potential”. Using this program target segments are generally selected that have estimates of no potential in these parameters. However, segments can be used that have “some potential” in one of the categories. A balance of the parameters is used in the selection as is known in the art. Further, the oligonucleotides are also selected as needed so that analogue substitution does not substantially affect function.

Phosphorothioate antisense oligonucleotides do not normally show significant toxicity at concentrations that are effective and exhibit sufficient pharmacodynamic half-lives in animals (Agrawal et al, 1996) and are nuclease resistant. Antisense induced loss-of-function phenotypes related with cellular development were shown for the glial fibrillary acidic protein (GFAP), for the establishment of tectal plate formation in chick (Galileo et al, 1991) and for the N-myc protein, responsible for the maintenance of cellular heterogeneity in neuroectodermal cultures (epithelial vs. neuroblastic cells, which differ in their colony forming abilities, tumorigenicity and adherence) (Rosolen et al, 1990; Whitesell et al, 1991). Antisense oligonucleotide inhibition of basic fibroblast growth factor (bFgF), having mitogenic and angiogenic properties, suppressed 80% of growth in glioma cells (Morrison, 1991) in a saturable and specific manner. Being hydrophobic, antisense oligonucleotides interact well with phospholipid membranes (Akhter et al, 1991). Following their interaction with the cellular plasma membrane, they are actively (or passively) transported into living cells (Loke et al, 1989), in a saturable mechanism predicted to involve specific receptors (Yakubov et al, 1989).

Instead of an antisense sequence as discussed hereinabove, ribozymes may be utilized. This is particularly necessary in cases where antisense therapy is limited by stoichiometric considerations (Sarver et al, 1990). Ribozymes can then be used that will target the same sequence. Ribozymes are RNA molecules that possess RNA catalytic ability (see Cech for review) that cleave a specific site in a target RNA. The number of RNA molecules that are cleaved by a ribozyme is greater than the number predicted by stoichiochemistry. (Hampel and Tritz, 1989; Uhlenbeck, 1987).

Ribozymes catalyze the phosphodiester bond cleavage of RNA. Several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA (sTRSV) (Sullivan, 1994; U.S. Pat. No. 5,225,347, columns 4-5). The latter two families are derived from viroids and virusoids, in which the ribozyme is believed to separate monomers from oligomers created during rolling circle replication (Symons, 1989 and 1992). Hammerhead and hairpin ribozyme motifs are most commonly adapted for trans-cleavage of mRNAs for gene therapy (Sullivan, 1994). The ribozyme type utilized in the present invention is selected as is known in the art. Hairpin ribozymes are now in clinical trial and are the preferred type. In general the ribozyme is from 30-100 nucleotides in length.

Modifications or analogues of nucleotides can be introduced to improve the therapeutic properties of the nucleotides. Improved properties include increased nuclease resistance and/or increased ability to permeate cell membranes.

Nuclease resistance, where needed, is provided by any method known in the art that does not interfere with biological activity of the antisense oligodeoxy-nucleotides, cDNA and/or ribozymes as needed for the method of use and delivery (Iyer et al, 1990; Eckstein, 1985; Spitzer and Eckstein, 1988; Woolf et al, 1990; Shaw et al, 1991). Modifications that can be made to oligonucleotides in order to enhance nuclease resistance include modifying the phosphorous or oxygen heteroatom in the phosphate backbone. These include preparing methyl phosphonates, phosphorothioates, phosphorodithioates and morpholino oligomers. In one embodiment it is provided by having phosphorothioate bonds linking between the four to six 3′-terminus nucleotide bases. Alternatively, phosphorothioate bonds link all the nucleotide bases. Other modifications known in the art may be used where the biological activity is retained, but the stability to nucleases is substantially increased.

The present invention also includes all analogues of, or modifications to, an oligonucleotide of the invention that does not substantially affect the function of the oligonucleotide. The nucleotides can be selected from naturally occurring or synthetic modified bases. Naturally occurring bases include adenine, guanine, cytosine, thymine and uracil. Modified bases of the oligonucleotides include xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thioalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogues of nucleotides can be prepared wherein the structure of the nucleotide is fundamentally altered and that are better suited as therapeutic or experimental reagents. An example of a nucleotide analogue is a peptide nucleic acid (PNA) wherein the deoxyribose (or ribose) phosphate backbone in DNA (or RNA) is replaced with a polyamide backbone, which is similar to that found in peptides. PNA analogues have been shown to be resistant to degradation by enzymes and to have extended lives in vivo and in vitro. Further, PNAs have been shown to bind more strongly to a complementary DNA sequence than a DNA molecule. This observation is attributed to the lack of charge repulsion between the PNA strand and the DNA strand. Other modifications that can be made to oligonucleotides include polymer backbones, cyclic backbones, or acyclic backbones.

The active ingredients of the pharmaceutical composition can include oligonucleotides that are nuclease resistant needed for the practice of the invention or a fragment thereof shown to have the same effect targeted against the appropriate sequence(s) and/or ribozymes. Combinations of active ingredients as disclosed in the present invention can be used including combinations of antisense sequences.

The antisense oligonucleotides (and/or ribozymes) and cDNA of the present invention can be synthesized by any method known in the art for ribonucleic or deoxyribonucleic nucleotides. For example, an Applied Biosystems 380B DNA synthesizer can be used. When fragments are used, two or more such sequences can be synthesized and linked together for use in the present invention.

The nucleotide sequences of the present invention can be delivered either directly or with viral or non-viral vectors. When delivered directly the sequences are generally rendered nuclease resistant. Alternatively the sequences can be incorporated into expression cassettes or constructs such that the sequence is expressed in the cell as discussed hereinbelow. Generally the construct contains the proper regulatory sequence or promoter to allow the sequence to be expressed in the targeted cell.

Negative dominant peptide refers to a partial cDNA sequence that encodes for a part of a protein, i.e., a peptide (see Herskowitz, 1987). This peptide can have a different function from the protein from which it was derived. It can interact with the full protein and inhibit its activity or it can interact with other proteins and inhibit their activity in response to the full protein. Negative dominant means that the peptide is able to overcome the natural proteins and fully inhibit their activity to give the cell different characteristics like resistance or sensitization to killing. For therapeutic intervention either the peptide itself is delivered as the active ingredient of a pharmaceutical composition or the cDNA can be delivered to the cell utilizing the same methods as for antisense delivery.

The present invention provides a method of providing an apoptotic regulating gene, an angiogenesis regulating gene or a hypoxia regulating gene by administering directly to a patient in need of such therapy utilizing gene therapy an expressible vector comprising expression control sequences operably linked to one of the sequences set forth in the group comprising SEQ ID NO:1; SEQ ID NO:2; SEQ ID NO:3; SEQ ID NO:4; SEQ ID NO:5; and SEQ ID NO:6.

Gene therapy as used herein refers to the transfer of genetic material (e.g., DNA or RNA) of interest into a host to treat or prevent a genetic or acquired disease or condition phenotype. The genetic material of interest encodes a product (e.g., a protein, polypeptide, peptide, functional RNA, antisense) whose production in vivo is desired. For example, the genetic material of interest can encode a hormone, receptor, enzyme, polypeptide or peptide of therapeutic value. Alternatively, the genetic material of interest encodes a suicide gene. For a review see, in general, the text “Gene Therapy” (August et al, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically re-implanted cells have been shown to express the transfected genetic material in situ.

In in vivo gene therapy, target cells are not removed from the subject. Rather, the genetic material to be transferred is introduced into the cells of the recipient organism in situ, that is, within the recipient. In an alternative embodiment, if the host gene is defective, the gene is repaired in situ (Culver, 1998). These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. An enhancer is generally any non-translated DNA sequence that works contiguously with the coding sequence (in cis) to change the basal transcription level dictated by the promoter. The expression vehicle can also include a selection gene as described hereinbelow.

Vectors can be introduced into cells or tissues by any one of a variety of known methods within the art. Such methods can be found generally described in Sambrook et al, (1989, 1992), Ausubel et al, (1989), Chang et al, (1995), Vega et al, (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor that includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic ganciclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation will not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention will depend on desired cell type to be targeted and will be known to those skilled in the art. For example, if breast cancer is to be treated then a vector specific for such epithelial cells would be used. Likewise, if diseases or pathological conditions of the hematopoietic system are to be treated, then a viral vector that is specific for blood cells and their precursors, preferably for the specific type of hematopoietic cell, would be used.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles that are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment. Administration can also be performed by, for example, intravenous or subcutaneous injection into the subject. Injection of the viral vectors into spinal fluid can also be used as a mode of administration, especially in the case of neuro-degenerative diseases. Following injection, the viral vectors will circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled in the art.

The pharmaceutical compositions containing the active ingredients of the present invention as described hereinabove are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the medical arts. The amount must be effective to achieve improvement including but not limited to improved survival rate or more rapid recovery, or improvement or elimination of symptoms and other indicators as are selected as appropriate measures by those skilled in the medical arts. The pharmaceutical compositions can be combinations of the active ingredients but will include at least one active ingredient.

In the method of the present invention, the pharmaceutical compositions of the present invention can be administered in various ways taking into account the nature of compounds in the pharmaceutical compositions. It should be noted that they can be administered as the compound or as a pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses may be single doses or multiple doses over a period of several days, but single doses are preferred.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver the pharmacological formulation orally or intravenously and retain the biological activity are preferred.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated and will vary from about 100 ng/kg of body weight to 100 mg/kg of body weight per day and preferably will be from 10 μg/kg to 10 mg/kg of body weight per day.

The present invention also provides a method of diagnosing the presence of ischemia in a patient including the steps of analyzing a bodily fluid or tissue sample from the patient for the presence or gene product of at least one expressed gene (up-regulated) as set forth in the group comprising SEQ ID NO:1; SEQ ID NO:2; SEQ ID NO:3; SEQ ID NO:4; SEQ ID NO:5; and SEQ ID NO:6 or proteins as set forth in SEQ ID NOs:7-11 and where ischemia is determined if the up-regulated gene or gene product is ascertained as described herein in Example 1. Examples 2-6 provide further experimental evidence for the diagnostic utility predicted in the present specification, i.e., that diagnosis of hypoxia or ischemia may be performed by identifying overexpression of gene RTP801. Example 7 provides further experimental evidence for the diagnostic utility of the antibodies predicted in the present specification, i.e., that diagnosis of hypoxia or ischemia may be performed by using antibodies to the proteins of the invention.

The bodily fluids may include tears, serum, urine, sweat or other bodily fluid where secreted proteins from the tissue that is undergoing an ischemic event may be localized. Additional methods for identification of the gene or gene product that can be used are immunoassays, such as ELISA or radioimmunoassays (RIA), which are known to those in the art particularly to identify gene products in the samples. Immunohistochemical staining of tissue samples is also utilized for identification. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521. Further, for identification of the gene, in situ hybridization, Southern blotting, single strand conformational polymorphism, restriction endonuclease fingerprinting (REF), PCR amplification and DNA-chip analysis using nucleic acid sequence of the present invention as primers can be used.

The above discussion provides a factual basis for the use of genes to regulate hypoxia and ischemia and thereby also apoptosis and angiogenesis. The methods used with and the utility of the present invention can be shown by the following non-limiting examples and accompanying figures.

### EXAMPLE 1

Methods:

Most of the techniques used in molecular biology are widely practiced in the art, and most practitioners are familiar with the standard resource materials that describe specific conditions and procedures. However, for convenience, the following paragraphs may serve as a guideline.

General Methods in Molecular Biology: Standard molecular biology techniques known in the art and not specifically described were generally followed as in Sambrook et al (1989), and in Ausubel et al (1989) particularly for the Northern analysis and in situ analysis and in Perbal, A Practical Guide to Molecular Cloning, John Wiley & Sons, New York (1988), and in Watson et al. Polymerase chain reaction (PCR) was carried out generally as in PCR Protocols: A Guide To Methods And Applications (1990).

Reactions and manipulations involving other nucleic acid techniques, unless stated otherwise, were performed as generally described in Sambrook et al (1989), and methodology as set forth in U.S. Pat. Nos. 4,666,828; 4,683,202; 4,801,531; 5,192,659 and 5,272,057 and incorporated herein by reference.

Additionally, in situ (in cell) PCR in combination with flow cytometry can be used for detection of cells containing specific DNA and mRNA sequences (Testoni et al, 1996).

General methods in immunology: Standard methods in immunology known in the art and not specifically described are generally followed as in Stites et al (1994) and Mishell et al (1980). Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al (1989).

Differential Analysis

For example C6 glioma cells or other appropriate cells, cell lines or tissues are grown under normal conditions (Normoxia) or under oxygen deprivation conditions (Hypoxia) generally for four to sixteen hours. The cells are harvested and RNA is prepared from the cytoplasmic extracts and from the nuclear fractions. Following the extraction of RNA, fluorescent cDNA probes are prepared. Each condition (for example 4 hours hypoxia and normoxia) is labeled with a different fluorescent dye. For example a probe can be composed of a mixture of Cy3-dCTP cDNA prepared from RNA extracted from hypoxic cells and with Cy5-dCTP cDNA prepared from RNA extracted from normoxic cells. The probes are used for hybridization to micro-array containing individually spotted cDNA clones derived from C6 cells that were exposed to hypoxia. Differential expression in measured by the amount of fluorescent cDNA that hybridizes to each of the clones on the array. Genes that are up-regulated under hypoxia will have more fluorescence of Cy3 than Cy5. The results show genes that are transcriptionally induced mRNA species that respond very fast to hypoxia.

Differential Display:

Reverse Transcription: 2 μg of RNA are annealed with 1 pmol of oligo dT primer (dT)₁₈ in a volume of 6.5 μl by heating to 70° C. for five minutes and cooling on ice. 2 μl reaction buffer (×5), 1 μl of 10 mM dNTP mix, and 0.5 μl of SuperScript II reverse transcriptase (GibcoBRL) is added. The reaction is carried out for one hour at 42° C. The reaction is stopped by adding 70 μl TE (10 mM Tris pH=8; 0.1 mM EDTA).

Oligonucleotides used for Differential Display: The oligonucleotides are generally those described in the Delta RNA Fingerprinting kit (Clonetech Labs. Inc.).

Amplification Reactions:

Each reaction is performed in 20 μl and contains 50 μM dNTP mix, 1 μM from each primer, 1× polymerase buffer, 1 unit expand Polymerase (Boehringer Mannheim), 2 μCi [α-³²P]dATP and 1 μl cDNA template. Cycling conditions are generally: three minutes at 95° C., then three cycles each of two minutes at 94° C., five minutes at 40° C., five minutes at 68° C. This is followed by 27 cycles of one minute each at 94° C., two minutes at 60° C., two minutes at 68° C. Reactions were terminated by a seven-minute incubation at 68° C. and addition of 20 μl sequencing stop solution (95% formamide, 10 mM NaOH, 0.025% bromophenol blue, 0.025% xylene cyanol).

Gel Analysis:

Generally 3-4 μl are loaded onto a 5% sequencing polyacrylamide gel and samples are electrophoresed at 2000 volts/40 milliamperes until the slow dye (xylene cyanol) is about 2 cm from the bottom. The gel is transferred to a filter paper, dried under vacuum and exposed to x-ray film.

Recovery of Differential Bands:

Bands showing any differential between the various pools are excised out of the dried gel and placed in a microcentrifuge tube. 50 μl of sterile H₂O are added and the tubes heated to 100° C. for five minutes. 1 μl is added to a 49 μl PCR reaction using the same primers used for the differential display and the samples are amplified for 30 cycles each of one minute at 94° C., one minute at 60° C. and one minute at 68° C. 10 μl is analyzed on agarose gel to visualize and confirm successful amplification.

Representational Difference Analysis

Reverse Transcription: as above but with 2 μg polyA+ selected mRNA.

Preparation of Double Stranded cDNA: cDNA from the previous step is treated with alkali to remove the mRNA, precipitated and dissolved in 20 μl H₂O. 5 μl buffer, 2 μl 10 mM dATP, H₂O to 48 μl and 2 μl terminal deoxynucleotide transferase (TdT) are added. The reaction is incubated 2-4 hours at 37° C. 5 μl oligo dT (1 μg/μl) were added and incubated at 60° C. for five minutes. 5 μl 200 mM DTT, 10 μl 10× section buffer (100 mM Mg Cl₂, 900 mM Hepes, pH 6.6) 16 μl dNTPs (1 mM), and 16 U of Klenow are added and the mixture incubated overnight at room temperature to generate ds cDNA. 100 μl TE is added and extracted with phenol/chloroform. The DNA is precipitated and dissolved in 50 ul H₂O.

Generation of Representations:

cDNA with DpnII is digested by adding 3 μl DpnII reaction buffer 20 V and DpnII to 25 μl cDNA and incubated five hours at 37° C. 50 μl TE is added and extracted with phenol/chloroform. cDNA is precipitated and dissolved to a concentration of 10 ng/μl.

Driver: 1.2 μg DpnII digested cDNA, 4 μl from each oligo and 5 μl ligation buffer ×10, are annealed at 60° C. for ten minutes. 2 μl ligase is added and incubated overnight at 16° C. The ligation mixture is diluted by adding 140 μl TE. Amplification is carried out in a volume of 200 μl using appropriate primer and 2 μl ligation product and repeated in twenty tubes for each sample. Before adding Taq DNA polymerase, the tubes are heated to 72° C. for three minutes. PCR conditions are as follows: five minutes at 72° C., twenty cycles of one minute each at 95° C. and three minutes at 72° C., followed by ten minutes at 72° C.

Every four reactions were combined, extracted with phenol/chloroform and precipitated. Amplified DNA is dissolved to a concentration of 0.5 μg/μl and all samples are pooled.

Subtraction: Tester DNA (20 μg) is digested with DpnII as above and separated on a 1.2% agarose gel. The DNA is extracted from the gel and 2 μg ligated to the appropriate oligos. The ligated Tester DNA is then diluted to 10 ng/μl with TE. Driver DNA is digested with DpnII and repurified to a final concentration of 0.5 μg/μl. Mix 40 μg of Driver DNA with 0.4 μg of Tester DNA. Extraction is carried out with phenol/chloroform and precipitated using two washes with 70% ethanol, resuspended DNA in 4 μl of 30 mM EPPS pH=8.0, 3 mM EDTA and overlaid with 35 μl mineral oil. Denature at 98° C. for five minutes, cool to 67° C. and add 1 μl of 5M NaCl to the DNA. Incubate at 67° C. for twenty hours. Dilute DNA by adding 400 μl TE.

Amplification: Amplification of subtracted DNA in a final volume of 200 μlis performed as follows: Buffer, nucleotides and 20 μl of the diluted DNA are added, heated to 72° C., and Taq DNA polymerase added. Incubate at 72° C. for five minutes and add appropriate oligo. Ten cycles each of one minute at 95C, three minutes at 70° C. are performed. Incubate ten minutes at 72° C. The amplification is repeated in four separate tubes. The amplified DNA is extracted with phenol/chloroform, precipitated and all four tubes combined in 40 μl 0.2×TE, and digested with Mung Bean Nuclease as follows: To 20 μl DNA 4 l buffer, 14 μl H₂O and 2 ul Mung Bean Nuclease (10 units/μl) added. Incubate at 30° C. for thirty-five minutes+First Differential Product (DPI).

Repeat Subtraction Hybridization and PCR Amplification at Driver: Differential ratio of 1:400 (DPII) and 1:40,000 (DPIII) using appropriate oligonucleotides. Differential products are then cloned into a Bluescript vector at the BAM HI site for analysis of the individual clones.

Differential Expression Using Gene Expression Micro-Array

Messenger RNA isolated as described hereinabove is labeled with fluorescent dNTP's using a reverse transcription reaction to generate a labeled cDNA probe. mRNA is extracted from C6 cells cultured in normoxia conditions and labeled with Cy3-dCTP (Amersham) and mRNA extracted from C6 cells cultured under hypoxia conditions is labeled with Cy5-dCTP (Amersham). The two labeled cDNA probes are then mixed and hybridized onto a microarray (Schena et al, 1996) composed of, for example, 2000 cDNA clones derived from a cDNA library prepared from C6 cells cultured under hypoxic conditions. Following hybridization, the microarray is scanned using a laser scanner and amount of fluorescence of each of the fluorescent dyes is measured for each cDNA clone on the micro-array, giving an indication of the level of mRNA in each of the original mRNA populations being tested. Comparison of the fluorescence on each cDNA clone on the micro-array between the two different fluorescent dyes is a measure for the differential expression of the indicated genes between the two experimental conditions.

In situ Analysis

In situ analysis is performed for the candidate genes identified by the differential response to exposure to hypoxia conditions as described above. The expression is studied in two experimental systems: solid tumors and hypoxic retina.

Solid tumors are formed by injections in mice of the original glioma cells used for the differential expression. The glioma cells form tumors which are then excised, sliced and used to individually measure expression levels of the candidate gene. The solid tumor model (Benjamin et al, 1997) shows that the candidate gene's expression is activated in tumors around the hypoxic regions that are found in the center of the tumor and are therefore hypoxia-regulated in vivo. Up regulation indicates further that the up-regulated gene can promote angiogenesis that is required to sustain tumor growth.

The hypoxia retina model measures expression levels in an organ that is exposed to hypoxia (ischemia) and directly mimics retinopathy. Hypoxia in the retina is created by exposing newborn rats to hyperoxia which diminishes the number of blood vessels in the retina (Alon et al, 1995). Upon transfer to normal oxygen levels, relative hypoxia is formed due to the lack of blood supply. The hypoxic retina is excised, sliced and used to monitor the expression of the candidate genes.

Results

Utilizing gene expression microarray analysis the genes set forth in SEQ ID NOs:1-6 were identified as being differentially expressed under hypoxia conditions.

As shown in the figures, differential expression under hypoxia conditions was observed. Northern analysis was performed with ³²P-dCTP labeled probes derived from the candidate genes. Two micrograms of mRNA were fractionated on formaldehyde-containing agarose gels, blotted onto a nitrocellulose membrane and hybridized to the labeled cDNA probes. To monitor the kinetics of expression as a result of hypoxia, mRNA was prepared from cells in normoxia, and 4 and 16 hours exposure to hypoxia conditions. The results of the analysis showed that all the genes (SEQ ID NOs:1-6) were induced by hypoxic conditions, confirming the results obtained by the gene expression microarray analysis.

In the in situ analysis using the solid tumor model SEQ ID NOs:1-6 were up-regulated, that is expressed. In the retina model SEQ ID NOs:1, 2 and 6 were found to be up-regulated.

SEQ ID NO:1 (RTP801) is the rat homolog of SEQ ID NO:2 (human RTP779). The protein sequences are SEQ ID NO:9 and SEQ ID NO:10 respectively. Neither of these genes has been reported in gene databases and both are expressed under hypoxic stress and are up-regulated in both of the in situ analyses. The expression of this gene was observed in the ovary where active apoptosis was occurring. Its regulation is HIF-1 dependent (Carmeliet et al, 1998) indicating further that the gene is associated with hypoxia-induced apoptosis. Some homology was found between the 3′UTR of RTP801 and the 5′UTR of a transcription factor (rat) pet-1 (Carmeliet et al, 1998; Spence et al, 1998; Fyodorov et al, 1998).

SEQ ID NO:3 (RTP241) is 1902 bp long, has not been reported in gene data bases and is expressed under hypoxic stress and up-regulated in both in situ analyses. The gene sequence has some homology with a yeast gene located upstream to the cox14 gene. The protein (SEQ ID NO:7) coded by the sequence contains a signal peptide region and therefore is secreted.

SEQ ID NO:4 (RTP220) is 4719 bp long, has not been reported in gene databases and is expressed under hypoxic stress and up-regulated in the tumor in situ analysis. The gene sequence has some homology with annilin from Drosophila. The protein sequence is set forth in SEQ ID NO:11.

SEQ ID NO:5 (RTP953/359) is a partial gene sequence that has not been found in gene databases and is expressed under hypoxic stress and up-regulated in both in situ analyses.

SEQ ID NO:6 (RTP971) is expressed under hypoxic stress and up-regulated in the tumor in situ analysis. The original analysis used the rat sequence. SEQ ID NO:6 is the human homolog and has greater than 90% homology with the rat sequence. Based on preliminary sequence analysis it appears to be the gene Neuroleukin or a member of that gene family. The gene has not been reported to be responsive to hypoxia conditions and is reported to be a new motility factor for astrocytes. The reported gene encodes a protein (SEQ ID NO:8, human homolog) that is identified as a glycolytic enzyme phosphohexose isomerase and as a survival factor for neurons (Niinaka et al, 1998; Watanabe et al, 1996).

Astrocyte motility is an important factor in the formation of blood vessels (angiogenesis) in brain and retina. Astrocytes can be considered oxygen level sensors as they respond under hypoxic conditions by secretion of angiogenic factors like WEGF. In an experiment primary astrocyte cultures were established and grown in vitro without serum and the astrocytes were immobile. However when conditioned medium from retinal cultures cultured under hypoxic conditions was added to the astrocyte cultures motility was observed. If the neuroleukin inhibitor (Obese et al, 1990), D-erythrose 4-phosphate (at 1.25 mM) was added clear indications of inhibition of motility were observed in the astrocyte cultures, indicating that the astrocyte motility (and stellation) was dependent on neuroleukin activity. Other results show that SEQ ID NO:6 is also HIF-1 dependent indicating further that the gene is associated with hypoxia-induced angiogenesis and apoptosis.

### EXAMPLE 2

### RTP801 is a p53-Independent, HIF-1-Responsive Gene

This experiment was performed to clarify the HIF-1 dependence of the RTP801/RTP779 gene orthologs and their independence of p53.

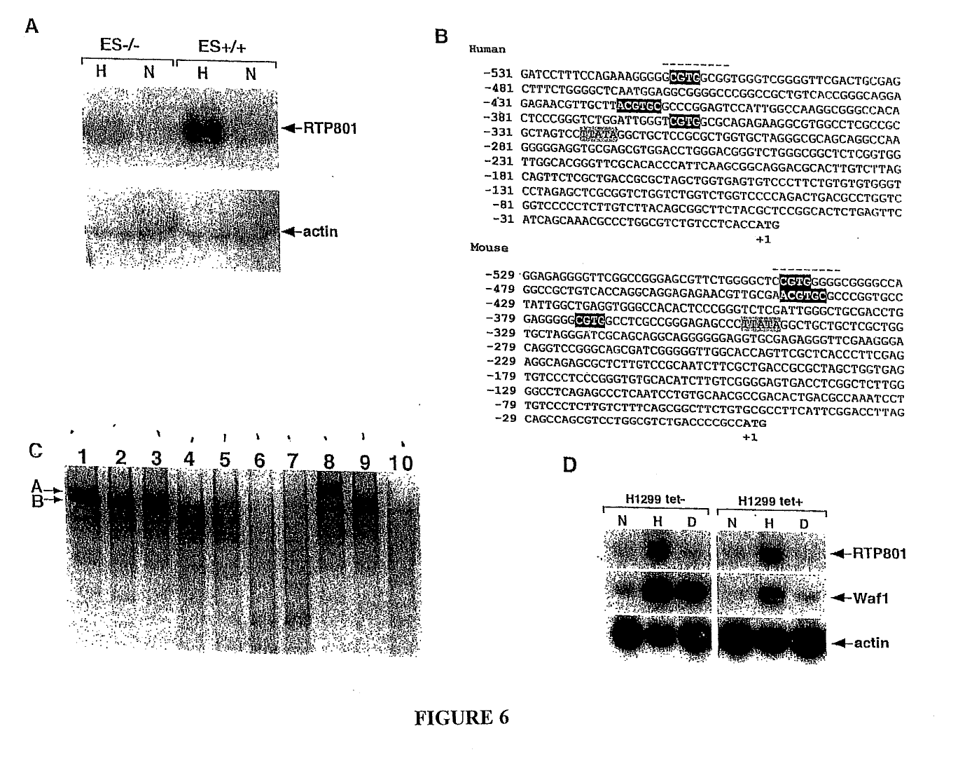

The kinetics of RTP801 response to hypoxia, as detected by microarray hybridization, resembled that of known HIF-1 targets, VEGF and glycolytic enzymes. This raised the possibility that RTP801 is an HIF-1-dependent gene as well. To test this hypothesis, we compared RTP801 mRNA induction by hypoxia in wild type and in HIF-1α −/−mouse embryonic stem (ES) cells (Carmeliet et al, 1998). As evident from FIG. 6A, unlike normal ES cells that displayed strong hypoxic stimulation of RTP801, the HIF-1α −/− ES cells failed to induce the RTP801 expression under similar conditions. This suggests that at least in ES cells, hypoxia-dependent stimulation of RTP801 is under the control of HIF-la.

To further confirm the HIF-1 dependence of RTP801, we tested whether its expression can be triggered by alternative stimuli known to activate the HIF-1 response. It was recently shown that treatment of cells with H₂O₂ is sufficient to promote HIF-1 stabilization (Chandel et al, 2000). Iron chelators, i.e., DFO, have also been shown to activate an hypoxia stress response pathway via HIF-1 (Zaman et al, 1999). As expected, the addition of either H₂O₂ or DFO to cells of various types elicited a rapid and strong up-regulation of RTP801.

HIF-1 stimulates transcription of its target genes by binding to a distinct nucleic acid motif named hypoxia responsive element (HRE). The putative regulatory regions immediately upstream of the first exons of mouse and human RTP801 orthologs were searched for the presence of HRE(s), using the Genomatix software. A mouse genomic clone was obtained from a mouse genomic λ phage library, while a human genomic clone containing the 5′ flanking region of RTP801 was identified by a database search (Acc AC006186). The HRE consensus sequence was previously described as either 5′-(G/C/T)ACGTGC(G/C)-3′ (Liu et al, 1995) or 5′-RCGTG-3′ (Wang et al, 1995). In both mouse and human DNA, three positionally conserved short HRE consensus motifs were detected within the 1000 bp preceding the first ATG codon (FIG. 6B). However, only one of them, located at positions −422 and −450 of human and mouse DNA, respectively, fit the extended consensus. Several additional potential HRE sites (the short consensus) were found within the more distant upstream region, but our data indicated that their positions were not conserved between mouse and human DNA.

To establish the direct involvement of HIF-1 in regulation of RTP801 transcription under hypoxic conditions, we performed electrophoretic mobility shift assays (EMSA) with the oligonucleotide containing the extended HRE consensus sequence derived from the mouse RTP801 promoter region (nt −454 to -434)-RTP801-HRE. A known HIF-1 binding oligonucleotide derived from the transferrin receptor gene promoter (TR-HRE) (Lok et al, 1999) and RTP801-specific oligonucleotide with the mutated core HRE sequence (RTP801-MHRE) were used as positive and negative controls, respectively. As evident from FIG. 6C, addition of ³²P-labeled TR-HRE oligonucleotide to nuclear extract of ES cells cultured under hypoxic conditions resulted in formation of two major complexes, A and B (lane 1). Two similarly migrating complexes were formed when RTP801-specific oligonucleotide was added to the same nuclear extract (lane 3); however, formation of complex A was abolished when RTP801-MHRE was used (lane 10), indicating the dependence of complex A on the presence of HRE core sequences. Formation of complex A was also inhibited when RTP801 radiolabelled probe was added to the nuclear extract from ES cells cultured in normoxic conditions (lane 2) or to nuclear extracts from HIF-1α −/− ES cells, regardless of whether they were maintained in hypoxia or normoxia (lanes 4 and 5, respectively). These results suggest that complex A is hypoxia-dependent and potentially contains HIF-1α. The specificity of formation of complex A on RTP801-HRE is proven by its competitive inhibition with an excess of the same non-labeled oligonucleotide (lane 6), while competitive inhibition with an excess of cold TR-HRE (lane 7), a known HIF-1-binding sequence, further supports the proposition that complex A formed with RTP801-HRE is HIF-1-dependent.

We next performed a supershift analysis of the observed complexes using the anti-HIF-1α antibodies. Their addition to the binding reaction with the radiolabeled RTP801-HRE resulted in a complete supershift of complex A (lane 8) whereas non-specific anti-Flag antibodies did not influence the mobility of any of the observed complexes (lane 9). Thus, the EMSA and supershift analyses have proven that hypoxia regulation of RTP801 is mediated via direct binding of HIF-1α-containing transcription complexes to its promoter.

p53 is known to be stabilized by forming a physical complex with HIF-1 (An et al, 1998) and to mediate HIF-1-dependent hypoxia-induced delayed neuronal death (Halterman et al, 1999). Therefore, we assessed whether hypoxic regulation of RTP801 is also p53-dependent. For this, we analyzed the response of RTP801 to hypoxia in several p53-negative (SCOV3, H1299, PC3) and p53-positive (MCF7, HT1080) cell lines. The results clearly indicate that hypoxic regulation of RTP801 is preserved regardless of the p53 status of the cells. An example of p53-independent activation of RTP801 transcription by hypoxia in H1299 cells is shown in FIG. 6D. Moreover, known p53-activating stimuli, like doxorubicin (FIG. 6D, lanes labeled “D”) failed to enhance the expression of RTP801. Results of similar experiments showed that UV and γ-irradiation also failed to enhance the expression of RTP801. Thus, while hypoxic regulation of RTP801 is HIF-1 dependent, it appears to be p53-independent.

The following conclusions can be drawn from this example. RTP801 is HIF-1 dependent. Focal areas of low oxygen tension (<2.0% O₂) are inherent to the biological processes of embryogenesis, wound repair, and carcinogenesis (Brown et al, 1998; Genbacev et al 1997; LaVan et al, 1990). A state of diminished free oxygen availability results when regional growth demands exceed the oxygen supply of the capillary bed (Brown et al, 1998). Under such conditions, an oxygen-sensing mechanism activates a transcription factor known as hypoxia-inducible factor-1 (HIF-1). The latter, in turn, switches on a series of genes participating in compensatory mechanisms that support the cell survival in a potentially lethal microenvironment. One group of HIF-1 target genes involved in the adaptive response facilitates O₂ delivery to oxygen-deprived tissues. It includes erythropoietin (stimulates production of erythrocytes); heme oxygenase 1 (mediates 0₂ binding to heme); vascular endothelial growth factor (triggers new vasculature formation); and inducible nitric oxide synthase (participates in local blood vessel dilation) (Semenza and Wang, 1992; Levy et al., 1995, Liu et al., 1995, Melillo et al., 1995; Lee et al., 1997). Another group of HIF-1-dependent genes acts to compensate for the inhibition of oxidative phosphorylation due to the lack of oxygen. It includes genes coding for glycolytic enzymes (i.e., lactate dehydrogenase, phosphoglyceromutase and others) and for glucose transporters (i.e., Glut1) (Firth et al., 1993; Semenza et al., 1994; Firth et al, 1994). Prolonged oxygen deprivation is detrimental for cells and may result in their death either through apoptotic or through necrotic mechanisms (reviewed in Lipton, 1999). Paradoxically, like the adaptive response to hypoxia, the hypoxia-dependent apoptosis was also shown to be HIF-1-dependent. Cells with genetically deleted HIF-1α were found to be resistant to hypoxia-induced apoptosis (Carmeliet et al., 1998), and HIF-1α was shown to mediate hypoxia-induced delayed neuronal death in the model of stroke (Halterman et al., 1999). While HIF-1-dependent genes participating in the adaptive response to hypoxia are widely characterized, genes mediating its proapoptotic function remain largely unknown. One of the proapoptotic genes, Nip3, was only recently characterized as HIF-1-dependent (Bruick, 2000). Expression of RTP801 can be triggered by alternative stimuli known to activate the HIF-1 response, e.g., H₂O₂ or DFO, an iron chelator. Possibly functional hypoxia responsive elements (HRE) have been located in both mouse and human DNA upstream to the RTP801 gene orthologs. Electrophoretic mobility shift assays and competitive inhibition experiments support the suggestion that a transcription complex formed at RTP801-HRE contains HIF-1. In addition, despite the fact that p53 is known to form a complex with HIF-1 and to be stabilized in this complex, results clearly indicate that hypoxic regulation of RTP801 is independent of p53 regulation. This evidence of HIF-1 dependence supports the association of the gene and its gene product with hypoxia and ischemia.

### EXAMPLE 3

### Inducible Expression of RTP801 in Neuron-Like Differentiated PC12 Cells Promotes Their Apoptosis and Sensitizes Them to Hypoxia- and H₂O₂-Triggered Cell Death

This experiment was performed to clarify the results of inducible expression of RTP801 on non-dividing, neuronal cells and to further understand the correlation of RTP801 expression and apoptosis in such cells.

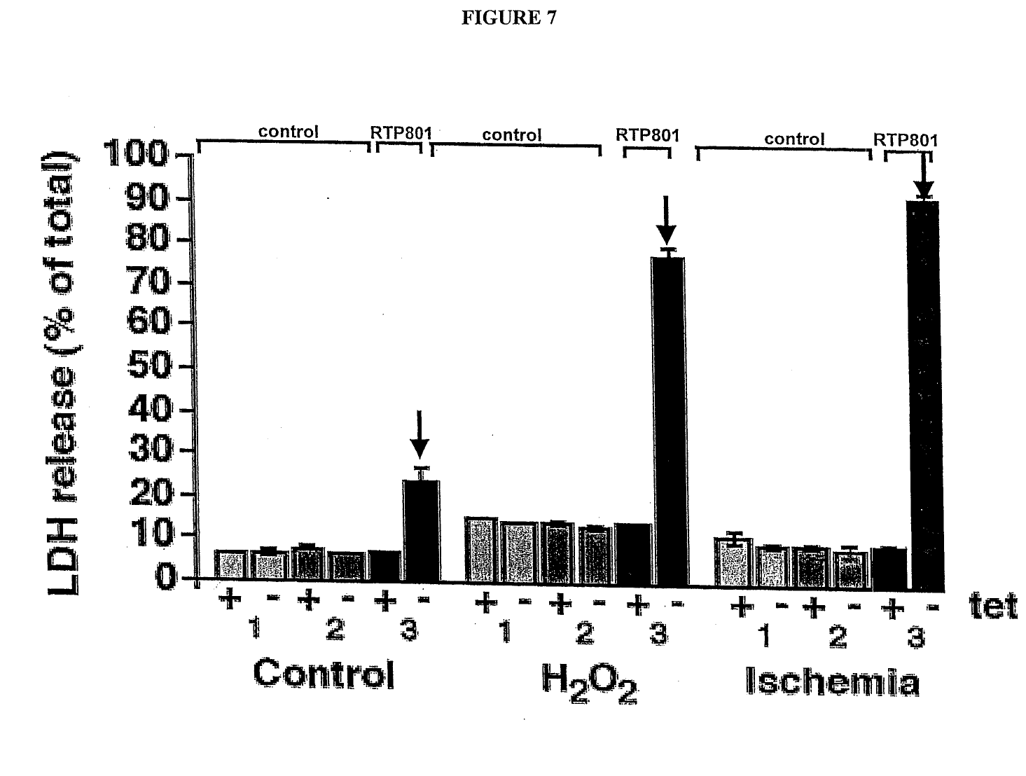

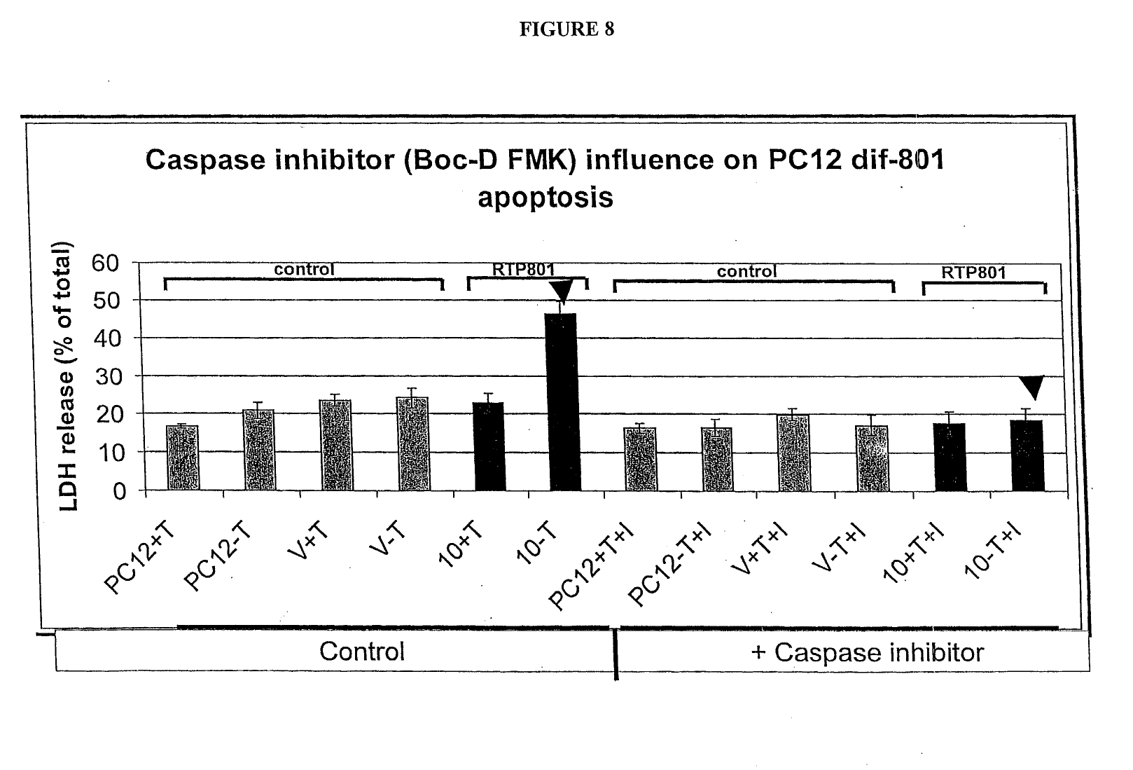

In light of the fact that up-regulation of endogenous RTP801 was observed in hypoxic neurons in vivo, we tested the influence of inducible expression of RTP801 on non-dividing neuron-like differentiated PC12 cells. A PC12-derived cell line expressing RTP801 under the control of a tetracycline-repressible promoter was generated. To promote neuronal differentiation, cells were treated with NGF (nerve growth factor) for five days in the presence of tetracycline. As a result, almost all the cells displayed the typical flattened morphology and outgrowth of processes. Seventy-two (72) hours after tetracycline removal, the cells were subjected to hypoxia/glucose deprivation or H₂O₂ treatments and the apoptotic response was evaluated after an additional 24 hours by measuring the LDH release (FIG. 7), Neutral Red uptake and appearance of apoptotic cells by staining the cell cultures with Apopercentage™ dye. Differentiated PC12 cells expressing exogenous RTP801 were significantly more sensitive to hypoxia and H₂O₂ compared to their control counterparts (FIG. 7). Moreover, induction of RTP801 expression by tetracycline removal was by itself sufficient to elicit the death response in these cells. Since RTP801-mediated cytotoxicity was completely abolished by addition of pancaspase inhibitor Boc-D (OMe)-FMK (FIG. 8), we concluded that induction of expression of RTP801 in differentiated PC12 cells leads to apoptosis via activation of caspases.

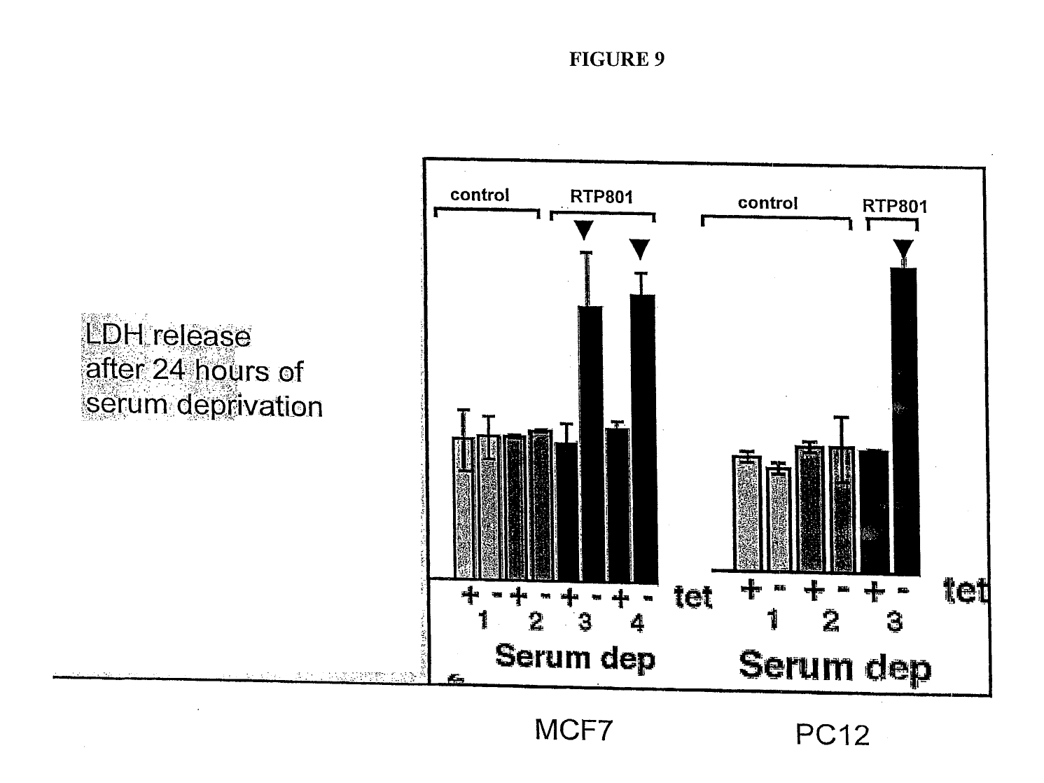

To test whether reduced concentration of serum may cause RTP801 to be proapoptotic, we transferred MCF7 and non-differentiated PC12 cells, in which expression of RTP801 was induced for 72 hours from the tetracycline-repressible promoter, into medium containing 0.1% serum. Remarkably, both MCF7 and PC12 cells that expressed exogenous RTP801 appeared much more sensitive to serum-deprivation than control cells (FIG. 9).

Thus, under certain conditions, expression of RTP801 may be detrimental to cells. Further experiments indicated that, in differentiated PC12 cells, the sensitivity to UV irradiation appeared to be unaffected by RTP801 overexpression.

The following conclusions can be drawn from this example. In vitro, overexpression of RTP801 sensitizes differentiated, non-dividing neuronal cells to apoptosis induced by ischemia or oxidative stress. There is indication that induction of expression of RTP801 in at least one type of differentiated cell leads to apoptosis via activation of caspases.

### EXAMPLE 4

### Liposomal Delivery of RTP801 to Mouse Lungs Elicits Apoptosis of Parenchymal Cells

This experiment was performed to assess the effect(s) of acute overexpression of RTP801 in vivo and to better understand the connection between overexpression of this gene and apoptosis. Overexpression, in preliminary experiments, was accomplished by liposomal delivery of the gene to mouse lung.

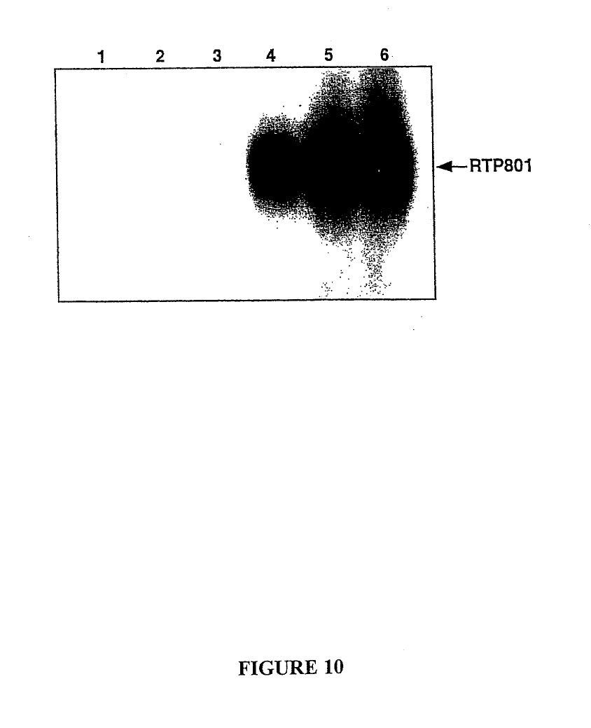

To assess the consequences of acute overexpression of RTP801 in vivo, cationic liposomes were used for the delivery of pcDNA3-RTP801 plasmid DNA into mouse lungs. Empty pcDNA3 vector and pcDNA3-p53 expression constructs served as negative and positive controls, respectively, for potential apoptotic response. Each of the three lipoplexes, containing 50 μg of plasmid DNA, was administered to 6 mice intravenously. Twenty-four (24) hours post-injection, the mice were sacrificed and their lungs removed for further evaluation. Northern blot analysis revealed high exogenous expression of RTP801 in RNA extracted from the lungs of pcDNA3-RTP801-injected mice but not in RNA from the lungs of control mice (FIG. 10)

In order to assess apoptosis in situ the technique of DNA end labeling staining (TUNEL) was utilized. The assay was performed using ApopTag® Peroxidase In Situ Apoptosis Detection kit (Intergen Company, NY, USA), according to the manufacturer's protocol. Parallel paraffin sections of lung samples were processed for TUNEL staining. It was noted that lungs of mice injected with RTP801-liposomes contained a large number of TUNEL-positive cells. The severity of the apoptotic response was in direct correlation with the intensity of RTP801 hybridization signal. Mice injected with the empty vector were generally TUNEL-negative. Only few TUNEL-positive cells were evident in the lungs of mice injected with p53 liposomes.

The following conclusions can be drawn from this example. Plasmid DNA carrying RTP801 was successfully delivered to the lungs of living mice using liposomes. Under these conditions, high exogenous expression of RTP801 in RNA extracted from lungs of experimental animals was observed, and was directly correlated with the severity of the accompanying apoptotic response.

### EXAMPLE 5

### The Expression of RTP801 Is Induced in the MCAO Model of Brain Ischemia (Stroke)

In this experimental system, MCAO (middle cerebral artery occlusion, a known animal model for the study of stroke), was utilized to study ischemia-dependent temporal and spatial patterns of RTP801 expression in cells of neuronal origin in vivo.

To further assess the hypoxia-dependent temporal and spatial pattern of RTP801 expression in cells of neuronal origin in vivo, we performed an in situ hybridization analysis in sections derived from a widely used rat model of brain ischemia (stroke) produced by MCAO. The injury of brain tissue in stroke results from a combination of pathophysiological processes that develop both within the ischemic core and within the surrounding peri-infarction area (penumbra) (Dirnagl et al, 1999).

The stroke model was prepared in a spontaneously hypertensive rat strain (SHR). A unilateral occlusion of the middle cerebral artery was accomplished using electrocoagulation. This led to a focal brain ischemia at the ipsilateral side of the brain cortex, leaving the contralateral side intact (control). Experimental animals were sacrificed 0.5, 1, 2, 12, 24, 48 and 72 hours after the operation (2 animals per time point). The brains were removed, fixed in formalin, embedded in paraffin and coronal sections were prepared for further use in in situ hybridization with ³⁵S-UTP labeled rat RTP801-, c-fos- and VEGF-specific sense and antisense riboprobes.

Rat RTP801 radioactive riboprobes were produced from the pBluescript-RTP801 vector (see above) using either T7 (antisense probe) or T3 (sense probe) as previously described (Komarova et al, 2000). In situ hybridization was performed according to a previously published protocol (Faerman et al, 1997). The exposed slides were developed in Kodak D-19 developer, fixed in Kodak fixer and counterstained with hematoxilin-eosin. The photomicrographs were taken using Zeiss Axioscop-2 microscope equipped with the Spot RT CCD camera (Diagnostic Instruments).

Coronal sections of rat brains fixed at different time points (from 30 minutes to 72 hours) after the MCAO procedure were prepared and were hybridized with the ³⁵S-labeled riboprobe complement to RTP801 mRNA. A c-fos-specific probe served as a positive control for delineation of the peri-infarction area at early time points following MCAO (Christensen et al 1993; Collaco-Moraes et al, 1994; Honkaniemi et al, 1997), while a VEGF-specific probe was used as a positive control for delineation of peri-infarction ischemic areas at later time points (Marti et al, 2000). In control brain sections, the RTP801 riboprobe produced a low intensity signal in cells of neuronal and glial origin. Permanent MCAO led to a rapid (within 30 minutes) intensification of the RTP801-specific signal in the ipsilateral cortical regions, suggesting up-regulation of RTP801 in response to stroke. The enhanced expression of RTP801 in neuronal and glial cells within the injured hemisphere was sustained at all analyzed post-insult time points, although a certain spatial redistribution of the hybridization signal was observed over the time course.

At early post-occlusion time points (0.5-2 hrs), the RTP801-specific signal was localized within distant peri-infarction areas that also displayed a prominent expression of c-fos. Expression of VEGF at this time point was still not evident. Twenty-four hours after the MCAO procedure was performed, accumulation of RTP801 messenger RNA occurred in VEGF-positive areas of the injured brain. It was significant within the eosinophilic neurons at the very boundary of the ischemic core although, in more distant cortex areas, RTP801-positive neurons looked morphologically normal. At 48 and 72 hours after the MCAO procedure, the RTP801-expressing neurons did not display any evident signs of ischemic injury. In addition, expression of RTP801 could also be detected in endothelial cells within the necrotic zone.

A complex expression pattern of RTP801 in the MCAO stroke model suggests that, in addition to hypoxia, its expression is regulated by other factors.

The following conclusions can be drawn from this example. Permanent MCAO led to a rapid (within 30 minutes) intensification of the RTP801-specific signal in the ipsilateral cortical regions, suggesting up-regulation of RTP801 in response to brain ischemia (stroke). Furthermore, a complex expression pattern of RTP801 in the MCAO model suggests that, in addition to hypoxia, its expression is regulated by other factors. However, the presence of RTP801 remains a valid diagnostic indicator of hypoxia.

### EXAMPLE 6

### The Expression of RTP801 in the MCAO Model of Stroke in Transgenic Mice

This experiment details the production of transgenic mice expressing RTP801 under the control of constitutive β-actin promoter. Mice with documented overexpression of RTP801 in brain cortex were subjected to MCAO in order to monitor the effect of RTP801 overexpression in an in vivo model of ischemic disease.

Transgenic FVBN mice, carrying RTP801, were bred for this series of experiments. Female RTP801 transgenic mice and their wild-type littermates, at 6-8 weeks of age, were both sham and MCAO operated. Twenty-four (24) hours following MCAO, brains were removed, fixed, and stained. Slices were prepared and imaged using a Spot Digital Camera. Analysis of infarct size in mouse brain was performed, using “ImagePro-Plus” computer software.

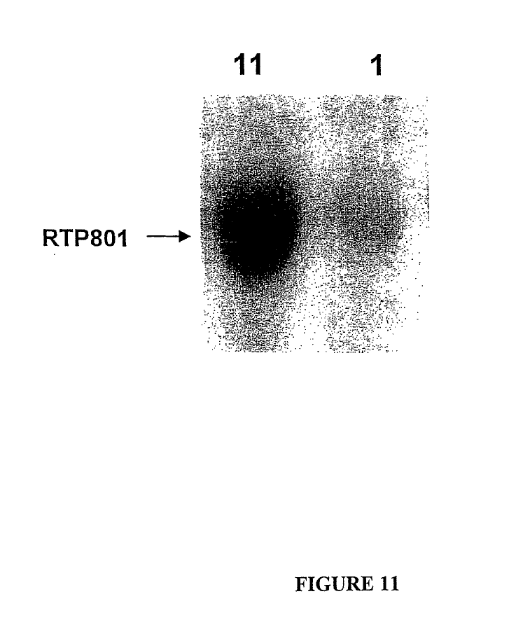

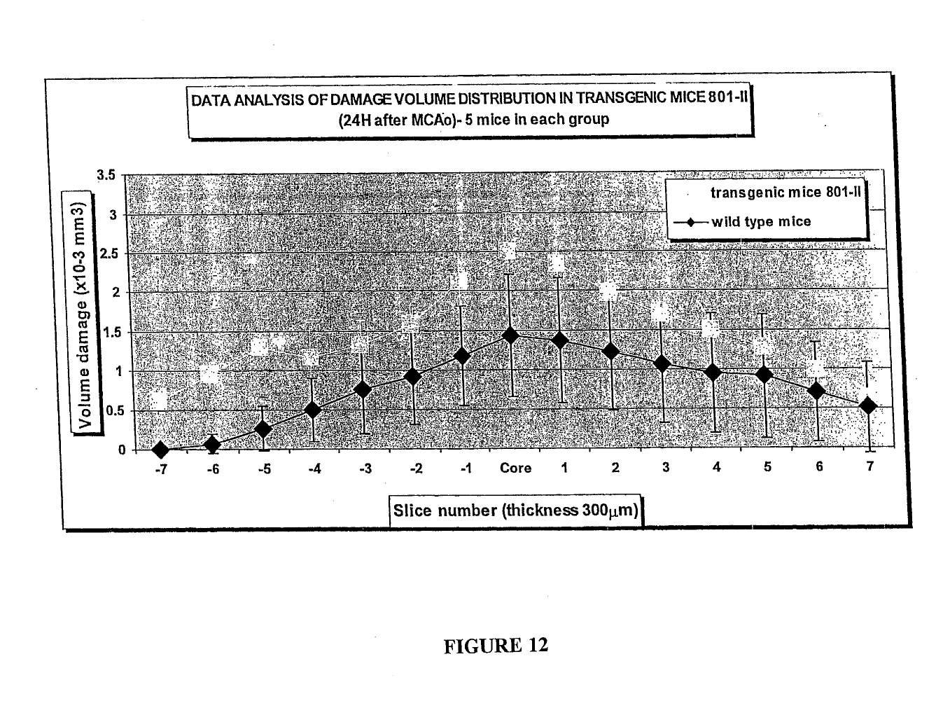

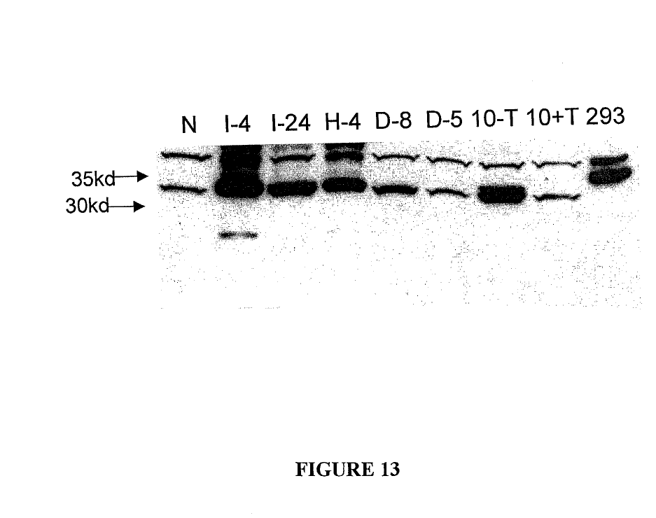

Results demonstrate that RTP801-transgenic mice that overexpress exogenous RTP801 in brain cortex (FIG. 11) display a dose-dependent increase of infarct size following MCAO, when compared to wild type mice (FIG. 12).

The following conclusions can be drawn from this example. Following MCAO, transgenic mice overexpressing RTP801 in their cortex displayed significantly larger infarct size (both within the core and within penumbra regions) compared to normal counterparts. These transgenic mice which overexpress RTP801 are, therefore, more susceptible to stroke. Furthermore, the transgenic mice that have now been produced can be made available for research purposes, in order to more fully understand the role of RTP801 in the process and progress of the stroke event.

### EXAMPLE 7

### Preparation of Rabbit Anti-RTP801-Protein Antibodies and Western Blot Analysis Thereof

Preparation of the Antibody: Rabbits were immunized against immunogen GST-801, using amino acids 1-230 of the protein (priming with 400 μg protein, followed by three boosts of 200 μg protein each). The serum was absorbed on immobilized GST, followed by absorption on immobilized bacterial proteins. The total serum IgG was then obtained by purification with immobilized protein.

Western Blot Analysis Using Rabbit Anti-RTP801 Antibodies: PC12 cells were exposed to ischemia (0.5% O₂ and 5% CO₂, in a glucose free medium), or H₂O₂ (0.5 mM) for 4 and 24 hours, or to the iron chelator deferoxamine mesylate (DFO) (300 ng/ml) for 5 and 8 hours. PC12, 801-tetracycline-induced clone 10 cells were grown for 72 hours either in the presence (10+T) or in the absence (10−T) of 1 μg/ml tetracycline. HEK293 cells were transiently transfected with RTP801 expression plasmid as positive control.

Whole cell protein extracts from the treated and control cells were prepared in RIPA lysis buffer (RIPA buffer: 50 mM Tris-HCl, pH 7.4; 150 mM NaCl; 1% deoxycholate Na; 1% NP-40; 0.1% SDS, 100 μM PMSF; 1 μM pepstatin A; 1 μM E64). The extracts were normalized for protein content, resolved on a 10% polyacrylamide-SDS gel and transferred to HybondP membrane (Amersham). The uniformity of protein loading on the gel was verified by subsequent Ponceau S staining of the membrane. The membrane was then blocked in PBS containing 10% milk and 0.1% Tween20 for 1 hour at room temperature and incubated with anti-RTP801 Ab at a concentration of 10 μg/ml for a further hour at room temperature in the same buffer. After washing, the membrane was incubated for 1 hour at 23° C. with the second antibody (anti-rabbit IgG 0.2 μg/ml) conjugated to horseradish peroxidase. The blots were processed using ECL-Plus Reagents (Amersham) according to the manufacturer's instructions.

Western analysis using 801 polyclonal antibody revealed a specific 30-35 kD band with maximal expression observed after 4 hr of ischemia (FIG. 14). In PC12 cells, with 801-tetracycline induced clone 10 grown in the absence of tetracycline (-Tet), a band of exogenous expression was detected. Note that the band indicating expression of the 801 gene in HEK293 cells transiently transfected with RTP801 expression plasmid, runs at approximately 35 kD, slightly heavier than the band detected by the 801 antibody produced in PC12 cells, possibly due to differences in post-translational modification.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### REFERENCES

Agrawal S, “Antisense oligonucleotides: towards clinical trials”, Trends Biotechnol 14(10):376-387 (1996)

Akhter et al, “Interactions of antisense DNA oligonucleotide analogs with phospholipid membranes (liposomes)”, Nuc Res 19:5551-5559 (1991)

Alon et al, “Vascular endothelial growth factor acts as a survival factor for newly formed retinal vessels and has implications for retinopathy of prematurity”, Nat Med 1(10):1024-1028 (1995)

An et al, “Multilineage embryonic hematopoiesis requires hypoxic ARNT activity”, Genes Dev 13:2478-2483 (1998)

August et al (Eds.), Gene Therapy Advances in Pharmacology Vol. 40, Academic Press (1997)

Ausubel et al, Current Protocols in Molecular Biology, John Wiley and Sons, Baltimore, Md. (1989)

Bellet et al, “Multiepitopic immunometric assay”, U.S. Pat. No. 5,011,771, issued Apr. 30, 1991

Benjamin, et al, “Conditional switching of vascular endothelial growth factor (VEGF) expression in tumors: induction of endothelial cell shedding and regression of hemangioblastoma-like vessels by VEGF withdrawal”, Proc Natl Acad Sci USA 94(16):8761-8766 (1997)

Borrebaeck, Antibody Engineering—A Practical Guide, W. H. Freeman and Co., 1992.

Bouck et al, “How tumors become angiogenic”, Adv Cancer Res 69:135-174 (1996)

Bradley et al, “Methods for the genetic modification of endogenous genes in animal cells by homologous recombination”, U.S. Pat. No. 5,614,396, issued Mar. 25, 1997

Brown et al, “The unique physiology of solid tumors: opportunities (and problems) for cancer therapy”, Cancer Res 58:1408-1416 (1998)

Bunn et al, “Oxygen sensing and molecular adaptation in hypoxia”, Physiol Rev 76:839-885 (1996)

Burke et al, “Preparation of Clone Libraries in Yeast Artificial-Chromosome Vectors” in Methods in Enzymology, Vol. 194, Guide to Yeast Genetics and Molecular Biology, eds. Guthrie et al, Academic Press, Inc., Chap. 17, pp. 251-270 (1991)

Byrne et al, “Multiplex gene regulation”, U.S. Pat. No. 5,221,778, issued Jun. 22, 1993

Calabretta et al, “Antisense strategies in the treatment of leukemias”, Semin Oncol 23:78 (1996)

Capecchi M R, “Altering the genome by homologous recombination” Science 244:1288-1292 (1989)

Capecchi et al, “Positive-negative selection methods and vectors”, U.S. Pat. No. 5,464,764, issued Nov. 7, 1995

Capecchi et al, “Cells and non-human organisms containing predetermined genomic modifications and positive-negative selection methods and vectors for making same”, U.S. Pat. No. 5,487,992, issued Jan. 30, 1996

Carmeliet et al, “Role of HIF-1 alpha in hypoxia-mediated apoptosis, cell proliferation and tumor angiogenesis”, Nature 394(6692):485-490 (1998)

Chandel et al, “Reactive oxygen species generated at mitochondrial complex III stabilize hypoxia-inducible factor-1-alpha during hypoxia: a mechanism of 0 2 sensing”, J Biol Chem 275:25130-25138 (2000)

Chang et al, Somatic Gene Therapy, CRC Press, Ann Arbor, Mich. (1995)

Christiansen et al, “Impairment of Fos protein formation in the rat infarct border zone by MK-801, but not by NBQX”, Acta Neurol Scand 87:510-515 (1993)

Collaco-Moraes et al, “Focal ischemia causes an extensive induction of immediate early genes that are sensitive to MK-801”, Stroke 25:1855-1861 (1994)

Cordel B, “Transgenic mice displaying the amyloid-forming pathology of Alzheimer's disease”, U.S. Pat. No. 5,387,742, issued Feb. 7, 1995

Crooke S T, “Progress in antisense therapeutics”, Hematol Pathol 2:59 (1995)

Davies et al, “Targeted alterations in yeast artificial chromosomes for inter-species gene transfer”, Nucleic Acids Res 20(11):2693-2698 (1992).

De Cant, Jr et al, “Variable flow implantable infusion apparatus”, U.S. Pat. No. 4,447,224, issued May 8, 1984

Dickinson et al, “High frequency gene targeting using insertional vectors”, Human Molecular Genetics, 2(8)1299-1302 (1993)

Dirnagl et al, “Pathobiology of ischaemic stroke: an integrated view’, Trends Neurosci 22(9):391-397 (1999)

Dor et al, “Ischemia-driven angiogenesis”, Trends Cardiovasc Med 7:289-294 (1997)

Dreyer W, “Immunological Reagent and Radioimmuno Assay”, U.S. Pat. No. 3,853,987, issued Dec. 10, 1974

Duff et al, “Insertion of a pathogenic mutation into a yeast artificial chromosome containing the human APP gene and expression in ES cells”, Research Advances in Alzheimer's Disease and Related Disorders, 1995.

Duke et al, “Cell Suicide in Health and Disease”, Sci Am 275(6):80-87 (1996)

Eckstein F, “Nucleoside Phosphorothioates”, Ann Rev Biochem 54:367-402 (1985)

Faerman et al, “Transgenic mice: production and analysis of expression”, Methods Cell Biol 52:373-403 (1997)

Felgner P L, “Nonviral Strategeies for Gene Therapy”, Sci Am, 276(6):102-106 (1997)

Ferrara J, “Therapeutic device for administering medicaments through the skin”, U.S. Pat. No. 4,486,194, issued Dec. 4, 1984

Firth et al, “Hypoxia and mitochondrial inhibitors regulate expression of glucose transporter-1 via distinct Cis-acting sequences”, J Biol Chem 270:29083-29090 (1993)

Frossard P, “Apo AI/CIII genomic polymorphisms predictive of atherosclerosis”, U.S. Pat. No. 4,801,531, issued Jan. 31, 1989

Fyodorov et al, “Pet-1, a novel ETS domain factor that can activate neuronal nAchR gene transcription”, J Neurobiol 34:151-163 (1998)

Gallagher et al, “Identification of p53 Genetic Suppressor Elements Which Confer Resistance to Cisplatin”, Oncogene 14:185-193 (1997)

Galileo et al, 1991. J. Cell. Biol., 112:1285.

Gearhart et al, “The Introduction and Expression of Large Genomic Sequences in Transgenic Animals”, International Publication WO 94/23049, published Oct. 13, 1994

Genbacev et al, “Regulation of human placental development by oxygen tension”, Science 277:1669-1672 (1997)

Gewirtz “Oligodeoxynucleotide-based therapeutics for human leukemias”, Stem Cells Dayt 11:96 (1993)

Goldberg et al, “Therapeutic ribozyme compositions and expression vectors”, U.S. Pat. No. 5,225,347, issued Jul. 6, 1993

Gordon J W, “Transgenic Animals”, Int Rev Cytol 115:171-229 (1989)

Gusella J, “Test for Huntington's disease”, U.S. Pat. No. 4,666,828, issued May 19, 1987

Gyory et al, “Control membrane for electrotransport drug delivery”, U.S. Pat. No. 5,169,383, issued Dec. 8, 1992

Haak et al, “Iontophoretic delivery method”, U.S. Pat. No. 5,167,616, issued Dec. 1, 1992

Halterman et al “Hypoxia-inducible factor-1-alpha mediates hypoxia-induced delayed neuronal death that involves p53”, J Neurosci 19:6818-6824 (1999)

Hanahan et al, “Patterns and Emerging Mechanisms of Angiogenic Switch During Tumorigenesis”; Cell 86:353-364 (1996)

Hanania et al, “Recent advances in the application of gene therapy to human disease”, Am J Med 99:537 (1995)

Harlow et al, Antibodies: A Laboratory Manual, Cold Spring Harbor Laboratory, Cold Spring Harbor, N.Y., (1988)

Harris D, “Implantable microinfusion pump system”, U.S. Pat. No. 4,487,603, issued Dec. 11, 1984

Herskowitz “Functional Inactivation of Genes By Dominant Negative Mutations”, Nature 329(6136):219-222 (1987).

Higuchi T, “Osmotic drug delivery system”, U.S. Pat. No. 4,439,196, issued Mar. 29, 1984

Holzmayer et al, “Isolation of Dominant Negative Mutants and Inhibitory Antisense RNA Sequences by Expression Selection of Random DNA Fragments”, Nucleic Acids Res 20(4):711-717 (1992)

Honkaniemi et al, “Expression of zinc finger immediate early genes in rat brain after permanent middle cerebral artery occlusion”, J Cereb Blood Flow Metab 17(6):636-646 (1997)

Huston et al, “Protein engineering of single-chain Fv analogs and fusion proteins” in Methods in Enzymology (J J Langone, ed.; Academic Press, New York, N.Y.) 203:46-88 (1991)

Huxley et al, “The human HPRT gene on a yeast artificial chromosome is functional when transferred to mouse cells by cell fusion”, Genomics 9:742-750 (1991)

Iyer et al, J Org Chem 55:4693-4699 (1990)

Jakobovits et al, “Germ-line transmission and expression of a human-derived yeast artificial chromosome”, Nature 362:255-261 (1993)

Johnson et al, “Construction of single-chain Fvb derivatives of monoclonal antibodies and their production in Escherichia coli”, Methods in Enzymol 203:88-99 (1991)

Johnstone et al, Immunochemistry in Practice, Blackwell Scientific Publications, Oxford, 1982

Komarova et al, “Different impact of p53 and p21 on the radiation response of mouse tissues”, Oncogene 19:3791-3798 (2000)

Krimpenfort et al, “Transgenic mice depleted in mature T-cells and methods for making transgenic mice”, U.S. Pat. No. 5,175,384, issued Dec. 29, 1992

Lamb et al, “Introduction and expression of the 400 kilobase precursor amyloid protein gene in transgenic mice”, Nature Genetics 5:22-29 (1993).

LaVan et al, “Oxygen and wound healing”, Clin Plast Surg 17:463-472 (1990).

Lavitrano et al, “Sperm cells as vectors for introducing foreign DNA into eggs: genetic transformation of mice”, Cell 57(5):717-723 (1989)

La Zor C, “Instrument for locating faults in aircraft passenger reading light and attendant call control system”, U.S. Pat. No. 4,475,196, issued Oct. 2, 1984

Leder et al, “Animal model for benign prostatic disease”, U.S. Pat. No. 5,175,383, issued Dec. 29, 1992

Leder et al, “Transgenic non-human mammals”, U.S. Pat. No. 4,736,866, issued Apr. 12, 1988

Lee et al, “Hypoxia-inducible factor-1 mediates transcriptional activation of the heme oxygenase-1 gene in response to hypoxia”, J Biol Chem 272:5375-5381 (1997)

Lefebvre-d'Hellencourt et al, “Immunomodulation by cytokine antisense oligonucleotides”, Eur Cytokine Netw 6:7 (1995)

Lev-Lehman et al, “Antisense Oligomers in vitro and in vivo” in Antisense Therapeutics, Cohen et al, eds (Plenum Press, New York) (1997)

Levy et al, “Transcriptional regulation of the rat vascular endothelial growth factor gene by hypoxia”, J Biol Chem 270:13333-13340 (1995)

Ling C-M, “Direct Radioimmunoassay for Antigens and Their Antibodies”, U.S. Pat. No. 3,867,517, issued Feb. 18, 1975

Lipton P, “Ischemic Cell Death in Brain Neurons”, Physiological Reviews 79(4):1431-1568) (1999)

Littman et al, “Transgenic Non-Human Animals Having Targeted Lymphocyte Transduction Genes”, International Publication WO 94/06908, published Mar. 31, 1994

Liu et al, “Hypoxia regulates vascular endothelial growth factor gene expression in endothelial cells. Identification of a 5′ enhancer”, Circ Res 77:638-643 (1995)

Lo C W, “Transformation by iontophoretic microinjection of DNA: multiple integrations without tandem insertions”, Mol Cell Biol 3:(10)1803-1814 (1983)

Lok et al, “Identification of a hypoxia response element in the transferrin receptor gene”, J Biol Chem 274(34):24147-24152 (1999)

Loke et al, “Characterization of oligonucleotide transport into living cells”, Proc Natl Acad Sci USA 86(10):3474-3478 (1989)

Mansour S L, “Gene targeting in murine embryonic stem cells: introduction of specific alterations into the mammalian genome”, Genet Anal Tech Appl 7(8):219-227 (1990)

Marshak et al, “Strategies for Protein Purification and Characterization. A laboratory course manual, CSHL Press (1996)

Marti et al, “Hypoxia-induced vascular endothelial growth factor expression precedes neovascularization after cerebral ischemia”, Am J Pathol 156:965-976 (2000)

Mayfield W B, “Medication infusion pump”, U.S. Pat. No. 4,447,233, issued May 8, 1984

McConnell H, “Process for Assaying for Biologically Active Molecules”, U.S. Pat. No. 3,850,578, issued Nov. 26, 1974

Melillo et al, “A hypoxia-responsive element mediates a novel pathway of activation of the inducible nitric oxide synthase promoter”, J Exp Med 182:1683-1693 (1995)

Mernaugh et al, “An overview of phage-displayed recombinant antibodies” in Molecular Methods In Plant Pathology (Singh et al, eds.), CRC Press Inc., Boca Raton, Fla. (1995) pp. 359-365

Miles L, “Universal reagent 2-site immunoradiometric assay using labelled anti (IgG)”, U.S. Pat. No. 4,034,074, issued Jul. 5, 1977

Mishell et al (eds), Selected Methods in Cellular Immunology, W. H. Freeman and Co., New York (1980)

Morrison R S, “Suppression of basic fibroblast growth factor expression by antisense oligodeoxynucleotides inhibits the growth of transformed human astrocytes”, J Biol Chem 266(2):728-734 (1991)

Mullis K, “Process for amplifying nucleic acid sequences”, U.S. Pat. No. 4,683,202, issued Jul. 28, 1987

Neuwelt, E A, “Method for the delivery of genetic material across the blood brain barrier”, U.S. Pat. No. 4,866,042, issued Sep. 12, 1989

Niinaka et al, “Expression and secretion of neuroleukin/phosphohexose isomerase/maturation factor as autocrine motility factor by tumor cells”, Cancer Res 58(12):2667-2674 (1998)

Obeso et al, “A Hemangioendothelioma-Derived Cell Line: Its Use as a Model for the Study of Endothelial Cell Biology”, Laboratory Investigation 83:259-264 (1990)

PCR Protocols: A Guide To Methods And Applications, Academic Press, San Diego, Calif. (1990)

Pearson et al, “Expression of the human β-amyloid precursor protein gene from a yeast artificial chromosome in transgenic mice”, Proc Natl Acad Sci USA 90:10578-10582 (1993)

Perbal, A Practical Guide to Molecular Cloning, John Wiley & Sons, New York (1988)

Piasio et al, “Reverse sandwich immunoassay”, U.S. Pat. No. 4,098,876, issued Jul. 4, 1978

Quertermous et al, “Cell specific gene regulators”, U.S. Pat. No. 5,288,846, issued Feb. 22, 1994

Radhakrishnan et al, “The automated synthesis of sulfur-containing oligodeoxyribonucleotides using 3H-1,2-Benzodithiol-3-One 1,1 Dioxide as a sulfur-transfer reagent”, J Org Chem 55:4693-4699 (1990)

Ranney D, “Endothelial envelopment drug carriers”, U.S. Pat. No. 4,925,678, issued May 15, 1990

Rosolen et al, “Antisense inhibition of single copy N-myc expression results in decreased cell growth without reduction of c-myc protein in a neuroepithelioma cell line”, Cancer Res 50(19):6316-6322 (1990)

Rothstein, “Targeting, disruption, replacement, and allele rescue: integrative DNA transformation in yeast” in Methods in Enzymology, Vol. 194, “Guide to Yeast Genetics and Molecular Biology”, Guthrie et al (eds), Academic Press, Inc., Chap. 19, pp. 281-301 (1991).

Rubenstein et al, “Antibody steric hindrance immunoassay with two antibodies”, U.S. Pat. No. 3,935,074, issued Jan. 27, 1976

Sambrook et al (Eds), Molecular Cloning: A Laboratory Manual, second edition, Cold Spring Harbor Laboratory Press, Cold Spring Harbor, (NY, 1989, 1992)

Sanders et al, “Delayed/sustained release of macromolecules”, U.S. Pat. No. 4,959,217, issued Sep. 25, 1990

Sarver et al, Gene Regulation and Aids, pp. 305-325 (1990)

Scanlon et al, “Oligonucleotides-mediated modulation of mammalian gene expression”, FASEB J 9:1288 (1995)

Schedl et al, “A yeast artificial chromosome covering the tyrosinase gene confers copy number-dependent expression in transgenic mice”, Nature 362:258-261 (1993).

Schena et al, “Parallel Human Genome Analysis: Microarray-based Expression Monitoring of 1000 genes”, Proc Natl Acad Sci USA 93(20):10614-10619 (1996)

Schuurs et al, “Detection and Determination of Haptens”, U.S. Pat. No. 3,879,262, issued Apr. 22, 1975

Schuurs et al, “Process for the Demonstration and Determination of Reaction Components Having Specific Binding Affinity for Each Other”, U.S. Pat. No. 3,791,932, issued Feb. 12, 1974

Schuurs et al, “Process for the Detection and Determination Of Specific Binding Proteins and Their Corresponding Bindable Substances”, U.S. Pat. No. 3,839,153, issued Oct. 1, 1974

Schuurs et al, “Process for the Demonstration and Determination of Low Molecular Compounds and of Proteins Capable of Binding These Compounds Specifically”, U.S. Pat. No. 3,850,752, issued Nov. 26, 1974

Schwartz et al, “Myogenic Vector Systems”, U.S. Pat. No. 5,298,422, issued Mar. 29, 1994

Semenza et al, “Transcriptional regulation of genes encoding glycolytic enzymes by hypoxia-inducible factor 1”, J Biol Chem 269:23757-23763 (1994)

Shaw et al, “Modified deoxyoligonucleotides stable to exonuclease degradation in serum”, Nucleic Acids Res 19:747-750 (1991)

Simons M, “Intron Sequence Analysis Method for Detection of Adjacent and Remote Locus Alleles as Haplotypes”, U.S. Pat. No. 5,192,659, issued Mar. 9, 1993

Smulson et al, “Method of detecting a predisposition to cancer by the use of restriction fragment length polymorphism of the gene for human poly(ADP-ribose) polymerase”, U.S. Pat. No. 5,272,057, issued Dec. 21, 1993

Spence et al, “Glucose metabolism in human malignant gliomas measured quantitatively with PET, 1-[C-11]glucose and FDG: analysis of the FDG lumped constant”, J Nucl Med 39(3):440-448 (1998)

Spitzer et al, “Inhibition of deoxynucleases by phosphorothioate groups in oligodeoxyribonucleotides”, Nucleic Acids Res 18:11691-11704 (1988)

Stites et al (eds), Basic and Clinical Immunology (8th Edition), Appleton & Lange, Norwalk, Conn. (1994)

Strauss et al, “Germ line transmission of a yeast artificial chromosome spanning the murine α₁ (I) collagen locus”, Science 259:1904-1907 (1993)

Testoni et al, “A new method of “in-cell reverse transcriptase-polymerase chain reaction” for the detection of BCR/ABL transcript in chronic myeloid leukemia patients”, Blood 87(9):3822-3827 (1996)

Thompson et al, “Mammals Lacking Expression of CD28 Transgenic”, International Publication WO 94/28123, published Dec. 8, 1994

Thompson et al, “Germ line transmission and expression of a corrected HPRT gene produced by gene targeting in embryonic stem cells”, Cell 56(2):313-321 (1989)

Trojanowski et al, “Modified avidin-biotin technique”, U.S. Pat. No. 5,281,521, issued Jan. 25, 1984

Uhlmann et al, “Antisense Oligonucleotides: A New Therapeutic Principle”, Chem Rev 90(4):543-584 (1990)

Ullman et al, “Fluorescence quenching with immunological pairs in immunoassays”, U.S. Pat. No. 3,996,345, issued Dec. 7, 1976

Uzgiris E, “Electrophoretic method of detecting antigen-antibody reaction”, U.S. Pat. No. 3,984,533, issued Oct. 5, 1976

van der Putten et al, “Efficient insertion of genes into the mouse germ line via retroviral vectors”, Proc Natl Acad Sci USA 82(18):6148-6152 (1985)

Vega et al, Gene Targeting, CRC Press, Ann Arbor, Mich. (1995)

Wadsworth et al, “Transgenic Animal Models for Alzheimer's Disease”, International Publication WO 93/14200, published Jul. 22, 1993

Wagner et al, “Genetic transformation of zygotes”, U.S. Pat. No. 4,873,191, issued Oct. 10, 1989

Wagner et al, “Virus-resistant transgenic mice”, U.S. Pat. No. 5,175,385, issued Dec. 29, 1992

Wagner, R W “Gene inhibition using antisense oligodeoxynucleotides”, Nature 372(6504):333-335 (1994)

Wagner et al, “Potent and selective inhibition of gene expression by an antisense heptanucleotide”, Nat Biotechnol 14(7):840-844 (1996)

Wands et al, “Immunoassay utilizing monoclonal high affinity IgM antibodies”, U.S. Pat. No. 4,879,219, issued Nov. 7, 1989

Wang et al, “Hypoxia-inducible factor 1 is a basic-helix-loop-helix-PAS heterodimer regulated by cellular O2 tension”, Proc Natl Acad Sci USA 92:5510-5514 (1995)

Watanabe et al, “Tumor cell autocrine motility factor is the neuroleukin/phosphohexose isomerase polypeptide”, Cancer Res 56(13):2960-2963 (1996)

Watson et al, Recombinant DNA, Scientific American Books, New York

Weinshank et al, “DNA encoding a human 5-HT_{1F} receptor, vectors, and host cells”, U.S. Pat. No. 5,360,735, issued Nov. 1, 1994

Whitesell et al, “Episome-generated N-myc antisense RNA restricts the differentiation potential of primitive neuroectodermal cell lines”, Mol Cell Biol 11:1360-1371 (1991)

Woolf et al, “The stability, toxicity and effectiveness of unmodified and phosphorothioate antisense oligodeoxynucleotides in Xenopus oocytes and embryos”, Nucleic Acids Res 18:1763-1769 (1990)

Wright et al, “Antisense Molecules and Their Potential For The Treatment Of Cancer and AIDS”, Cancer J 8:185-189 (1995)

Yakubov et al, “Mechanism of oligonucleotide uptake by cells: involvement of specific receptors?”, Proc Natl Acad Sci USA 86(17):6454-6458 (1989)

Zaman et al, “Protection from oxidative stress-induced apoptosis in cortical neuronal cultures by iron chelators is associated with enhanced DNA binding of hypoxia-inducible factor-1 and ATF-1/CREB and increased expression of glycolytic enzymes p21(waf12/cip1), and erythropoietin”, J Neurosci 19(22):9821-9830 (1999)

AC006186: Homo sapiens chromosome 10 clone CRI-JC2048 map 10q22.1 sequencing in progress; LOCUS: AC006186; submitted Aug. 30, 2001

What is claimed is:

1. An isolated nucleic acid molecule comprising a sequence encoding a polypeptide having an amino acid sequence selected from the group consisting of SEQ ID NO:9 and SEQ ID NO:10.

2. An isolated nucleic acid molecule in accordance with claim 1 comprising a sequence encoding a polypeptide having an amino acid sequence selected from the group consisting of SEQ ID NO:9.

3. An isolated nucleic acid molecule in accordance with claim 1 comprising a sequence encoding a polypeptide having an amino acid sequence selected from the group consisting of SEQ ID NO:10.

4. An isolated nucleic acid molecule in accordance with claim 1 having the sequence of SEQ ID NO:1 or SEQ ID NO:2.

5. An isolated nucleic acid molecule in accordance with claim 4 having the sequence of SEQ ID NO:1.

6. An isolated nucleic acid molecule in accordance with claim 4 having the sequence of SEQ ID NO:2.

7. An antibody which specifically binds to a polypeptide having an amino acid sequence selected from the group consisting of SEQ ID NO:9 and SEQ ID NO:10.

8. An antibody in accordance with claim 7 which specifically binds to a polypeptide having an amino acid sequence selected from the group consisting of SEQ ID NO:9.

9. An antibody in accordance with claim 7 which specifically binds to a polypeptide having an amino acid sequence selected from the group consisting of SEQ ID NO:10.

10. An antibody in accordance with claim 7 which is a monoclonal or a polyclonal antibody.

11. An antibody in accordance with claim 10 which is conjugated to a detectable moiety.

12. A method for the treatment of a subject in need of treatment for hypoxia or ischemia-related disease comprising administering to said subject a therapeutically effective amount of an antagonist of a protein having a sequence as set forth in SEQ ID NO:10, or an analogue thereof.

13. The method of claim 12, wherein the hypoxia or ischemia-related disease is stroke.
